(19) United States
(12) Patent Application Publication
(10) Pub. No.: US 2009/0318627 A1
(43) Pub. Date: Dec. 24, 2009

(54) SOLID PHASE SYNTHESIS OF ACRIDINIUM DERIVATIVES

(75) Inventors: Anand Natrajan, Manchester, NH (US); David Sharpe, Foxboro, MA (US); Jim Costello, Boston, MA (US); Qingping Jiang, East Walpole, MA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Healthcare Diagnostics Inc., Tarrytown, NY (US)

(21) Appl. No.: 12/278,621

(22) PCT Filed: Feb. 6, 2007
(86) PCT No.: PCT/US07/61696

§ 371 Date: Aug. 7, 2008

Related U.S. Application Data
(60) Provisional application No. 60/771,059, filed on Feb. 7, 2006.

(51) Int. Cl. C08G 63/91; C07D 219/00; C07J 43/00; C07K 2/00; C07H 21/00

(52) U.S. Cl. 525/418; 546/102; 540/2; 530/300; 536/22.1

(57) ABSTRACT

Acridinium-functionalized solid-phase supports and methods for making acridinium-functionalized solid-phase supports are disclosed. The acridinium-functionalized solid-phase supports comprise a solid phase support linked to a chemiluminescent substituted acridinium compound through a linker group covalently attached to the nitrogen atom of the acridinium nucleus and the solid phase support as exemplified in FIG. 1.

### FIELD OF INVENTION

The present invention relates generally to the solid phase synthesis of acridinium compounds and their conjugates.

### BACKGROUND OF INVENTION

Chemiluminescent acridinium esters (AEs) are extremely useful labels that have been used extensively in immunoassays and nucleic acid assays. A recent review, Pringle, M. J., Journal of Clinical Ligand Assay vol. 22, pp. 105-122 (1999), summarizes past and current developments in this class of chemiluminescent compounds.

McCapra, F. et al., Tetrahedron Lett. vol. 43, pp. 3167-3172 (1964) and Rahut et al. J. Org. Chem. vol. 301, pp. 3587-3592. (1965) disclosed that chemiluminescence from the esters of acridinium salts could be triggered by alkaline peroxide. Since these early studies, interest in acridinium compounds has increased because of their utility as chemiluminescent labels. The application of the acridinium ester 9-carboxyphenyl-N-methylacridinium bromide in an immunoassay was reported by Simpson, J. S. A. et al., Nature vol. 279, pp. 646-647 (1979). This acridinium ester is quite unstable owing to hydrolysis of the ester linkage between the acridinium ring and the phenol thereby limiting its commercial utility unless special precautions are taken to protect the acridinium ester from hydrolysis. For example, U.S. Pat. No. 4,950,613 to Arnold et al. shows that the hydrolytic stability of unstable acridinium esters can be alleviated somewhat with certain additives. A novel way to protect the acridinium ester from hydrolysis was described by Nelson et al. Biochemistry vol. 35, pages 8429-8438, 1996 in nucleic acid assays where a nucleic acid labeled with an unstable acridinium ester was protected from hydrolysis when the labeled nucleic acid bound to its target. This protection was thought to arise from binding of the acridinium ester in the DNA duplex in a water-poor environment.

Different strategies for increasing the hydrolytic stability of acridinium compounds by altering their structures have been described. Law et al., Journal of Bioluminescence and Chemiluminescence, vol. 4, pp. 88-89 (1989) describes the introduction of two methyl groups to flank the acridinium ester moiety to stabilize the ester bond through steric effects. The resulting acridinium ester, DMAE-NHS [2′,6′-dimethyl-4′-(N-succinimidyloxycarbonyl)phenyl-10-methylacridinium-9-carboxylate], was found to have the same light output as an acridinium ester lacking the two methyl groups but was significantly more resistant to hydrolysis. The structure of DMAE-NHS is shown below.

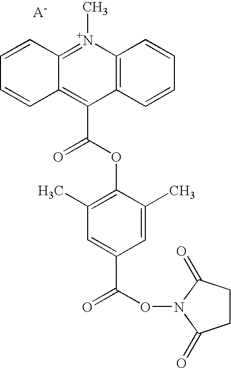

U.S. Pat. Nos. 4,918,192 and 5,110.932 describe DMAE and its applications. U.S. Pat. No. 5,656,426 by Law et al. discloses a hydrophilic version of DMAE termed NSP-DMAE-NHS ester where the methyl group on the acridinium ring nitrogen is replaced with a sulfopropyl group, as shown below:

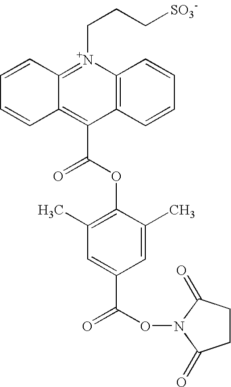

U.S. Pat. No. 6,664,043 B2 to Natrajan et al. discloses NSP-DMAE derivatives with hydrophilic modifiers attached to the phenol. The structure of one such compound is illustrated below.

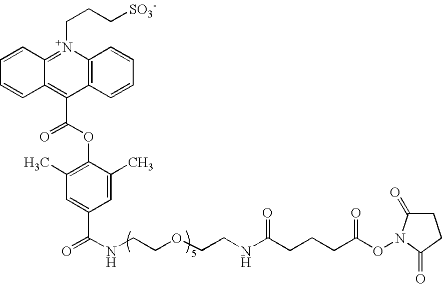

In this compound a diamino hexa(ethylene) glycol (HEG) moiety was attached to the phenol to increase the aqueous solubility of the acridinium ester. A glutarate moiety was appended to the end of HEG and was converted to the NHS ester to enable labeling of various molecules. Both DMAE and NSP-DMAE and their derivatives are currently used in Siemens Medical Solutions Diagnostics' ACS:180® and Advia Centaur® immunoanalyzers.

A different class of stable chemiluminescent acridinium compounds has been described by Kinkel et al., Journal of Bioluminescence and Chemiluminescence vol. 4, pp. 136-139 (1989) and Mattingly, Journal of Bioluminescence and Chemiluminescence vol. 6, pp. 107-114 (1991) and U.S. Pat. No. 5,468,646. In this class of compounds, the phenolic ester linkage is replaced by a sulfonamide moiety, which is reported to impart hydrolytic stability without compromising the light output. In acridinium esters, the phenol is the ‘leaving group’ whereas in acridinium sulfonamides, the sulfonamide is the ‘leaving group’ during the chemiluminescent reaction with alkaline peroxide. An example of a sulfonamide functionalized acridinium is shown below where R₁ and R₂ represent alkyl or aryl groups:

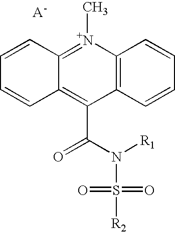

Solid phase organic synthesis has gained enormous popularity in the last decade for the rapid construction of a wide range of interesting molecules. A recent book on this subject Organic Synthesis on Solid Phase by F. Z. Dorwald, Wiley-VCH, 2001, reviews in detail commonly used solid phases, linker chemistry and synthetic reactions that have been described in the literature. In contrast to solution phase synthesis where all reactants are dissolved in a solvent, solid phase synthesis employs a solid support to which at least one of the reactants is covalently bound. Solid phase synthesis often has the advantage of speed and can be used to build vast ‘libraries’ of compounds, which can then be screened for biological activity. Two common approaches that are used for the construction of compound libraries (commonly referred to as combinatorial chemistry) are the so-called ‘parallel’ and ‘split-pool’ approaches. In parallel library synthesis, a compound with a discrete structure is synthesized on a solid phase. At the end of the synthesis, which can involve multiple steps, the structure of the compound on the solid phase is fixed and known. In split-pool synthesis, several variants of a structure are synthesized on a solid phase. Typically, the solid phase will have a mixture of several compounds with different structures. The split-pool approach has the advantage that for a given number of synthetic transformations, larger compound libraries can be synthesized. (see Wilson & Czarnik in Combinatorial Chemistry: Synthesis and Application, John Wiley & Sons Inc., 1997).

The compound libraries generated from parallel and split-pool syntheses can be screened for biological activity. From such studies, important mechanistic and structural information concerning the biological system can be elucidated. For example, screening a library of structurally related compounds for binding to an enzyme or an antibody, one can gain knowledge about the binding site of the antibody or enzyme. If inhibiting the activity of the enzyme has some therapeutic utility, then such screening studies can identify new medicines. When screening a compound library from a split-pool synthesis, usually an additional step called ‘de-convolution’ must be performed to identify either a subset of set of structures or, a discrete structure responsible for the observed biological activity. De-convolution is often performed by ‘tagging’ the solid phase with other molecules whose presence can be deduced independently.

Solid phase synthesis also has other advantages. Because reactions occur on a solid phase, excess reagents can be removed by filtration thus minimizing the number of purification steps that have to be performed thereby saving time, expensive chromatography supports and solvents. Moreover, reactions on a solid phase often exhibit altered reactivity and/or stability patterns that can be very useful as will be discussed in the present invention. Also, for the synthesis of synthetic peptides and nucleic acids, solid phase synthesis has really no useful solution-phase counterpart. The assembly of long peptides and nucleic acids would be next to impossible without solid phase synthesis.

Acridinium compounds are commonly used as chemiluminescent labels in immunoassays for small and large molecules that are often commonly referred to as analytes. When a solid phase such as a particle or microtiter plate is used in the assay, the assay is then commonly referred to as a solid-phase assay or heterogeneous assay. Heterogeneous assays for small molecules are also called ‘competitive assays’. Typically, in a competitive assay, a conjugate is made of the analyte of interest and a chemiluminescent or fluorescent label by covalently linking the two molecules. The small molecule analyte can be used as such or its structure can be altered prior to conjugation to the label. The analyte with the altered structure is called an analog. It is often necessary to use a structural analog of the analyte to permit the chemistry for linking the label with the analyte. Sometimes a structural analog of an analyte is used to attenuate or enhance its binding to a binding molecule such an antibody. Such techniques are well known in the prior art. The antibody or a binding molecule to the analyte of interest is often immobilized on a solid phase either directly or through a secondary binding interaction such as the biotin-avidin system. Such systems are well known in the prior art.

The concentration of the analyte in a sample can be deduced in a competitive assay by allowing a sample suspected of containing the analyte and the analyte-label conjugate to compete for a limited amount of binding molecule immobilized on a solid phase. As the concentration of analyte in a sample increases, the amount of analyte-label conjugate captured by the binding molecule on the solid phase decreases. By employing a series of ‘standards’, that is, known concentrations of the analyte, a dose-response curve can be constructed where the signal from the analyte-label conjugate captured by the binding molecule on the solid phase is inversely correlated with the concentration of analyte. Once a dose-response curve has been devised in this manner, the presence and concentration of the same analyte in an unknown sample can be deduced by comparing the signal obtained from the unknown sample with the signal in the dose-response curve.

Acridinium compound conjugates of analytes, especially small molecule analytes, called tracers are used in conjunction with antibodies for devising immunoassays for these analytes. The tracers are normally synthesized using solution phase synthesis techniques examples of which can be found in U.S. Pat. No. 5,656,426. The use of solid phase synthesis for the assembly of such acridinium ester structures that are the subject of the present invention has not been described in the prior art.

Solid phase synthesis of tracers, such as the attachment of various dyes, including an acridinium sulfonamide, to solid phases, has been described in articles such as M. Adamczyk et al./Bioorg. Med. Chem. Lett. 9 (1999) 217-220. However, there are substantial differences between that chemistry and what is illustrated here in the present invention. For example, in M. Adamczyk et al. solid phase-attached dyes are reacted with various nucleophiles which subsequently release the dye conjugates from the solid phase. Thus, this chemistry is largely restricted to a single displacement reaction on the solid phase and is not amenable to combinatorial synthesis. The chemistry of the present invention enables multiple synthetic transformations on the solid phase and the chemistry that is employed for attachment of the acridinium ester to the solid phase allows for the synthesis of libraries of AE-conjugates previously unavailable.

In light of the interesting properties of chemiluminescent acridinium compounds, there is a need in the art for improved synthetic methodologies for preparing acridinium compound derivatives and conjugates.

It is therefore an object of the invention to provide methods for solid phase synthesis of acridinium compound derivatives and conjugates.

It is another object of the invention to provide acridinium-functionalized solid phase supports as reagents for solid phase synthesis of acridinium compound derivatives and conjugates.

It is yet another object of the invention to provide acridinium-functionalized solid phase supports as chemiluminescent reagents for immunoassays.

### SUMMARY OF THE INVENTION

In accordance with the foregoing objectives and others, the present invention provides an acridinium-functionalized solid-phase support comprising a solid phase support having immobilized thereon a chemiluminescent acridinium compound. The chemiluminescent acridinium compound comprises a linker group covalently attached to the nitrogen atom of the acridinium nucleus and the solid phase support. In some implementations, the acridinium-functionalized solid-phase support has the structure of formula I:

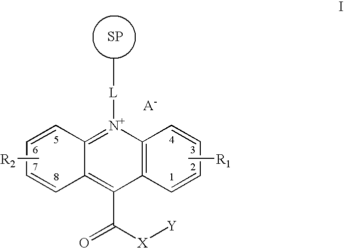

wherein,
L is a sulfonate ester or carboxylate ester linker group between the nitrogen of the acridinium nucleus and said solid phase support;
R₁ represents a substituent at one or more of carbon atoms 1-4 and R₂ represents a substituent at one or more of carbon atoms 5-8; R₁ and R₂ being independently selected at each occurrence from the group consisting of hydrogen, substituted or unsubstituted alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof;
X is O, S, or NR^{a}; where R^{a} is —SO₂—R′, R′ being selected from the group consisting of, substituted or unsubstituted, branched or straight chain alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof;
   and in the case where X is O or S, Y is a substituent of the formula:

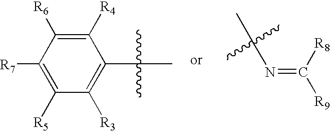

wherein at least one of R₃, R₄, R₅, R₆, R₇, R₈, and R₉ is independently a group -Q-R₁₀, wherein R₁₀ is a group comprising one or more reactive functional groups; where Q represents a bond or a functional group selected from the group consisting of branched or straight-chain alkyl, alkenyl, alkynyl, substituted or unsubstituted aryl, alkyl-aryl, and aryl-alkyl, optionally containing one or more heteroatoms selected from the group consisting of oxygen, nitrogen, phosphorous, sulfur, halogen, and combinations thereof;
and wherein any of R₃, R₄, R₅, R₆, R₇, R₈, and R₉ which are not a group -Q-R₁₀ are substituents independently selected from the group consisting of substituents defined above for R₁ and R₂, hydroxyl, halogen, alkyl, alkenyl, alkynyl, aryl, alkyl-aryl, or aryl-alkyl, —OR^{b}, —SR^{b}, cyano, carboxyl, —(C═O)—OR^{b}, —NR^{b}R^{c}, or —(C═O)—NR^{b}R^{c}, where R^{b} and R^{c} are independently selected from the substituents defined above for R₁ and R₂;
   and in the case where X is NR^{a}, Y is a group -Q-R₁₀ as defined above;
A⁻ is a counter ion selected from the group consisting of CH₃SO₄⁻, FSO₃⁻, CF₃SO₄⁻, C₄F₉SO₄⁻, CH₃C₆H₄SO₃⁻, halide, CF₃COO⁻, CH₃COO⁻, and NO₃⁻; and
   SP represents a solid phase support selected from the group consisting of polystyrene Wang resin, a paramagnetic particle, a latex particle, and a microtiter plate.

In another aspect of the invention, a method is provided for the solid phase synthesis of acridinium compound derivatives or conjugates comprising the steps of: (a) providing an acridinium-functionalized solid phase support comprising a solid phase support having immobilized thereon a chemiluminescent acridinium compound; wherein the substituted acridinium compound comprises a linker group covalently attached to the nitrogen atom of the acridinium nucleus and the solid phase support; (b) performing one or more synthetic transformations on the acridinium compound to provide a derivative or conjugate of the acridinium compound; and (c) cleaving the derivative or conjugate of the acridinium compound from the solid phase support. In some implementations of the inventive method, the acridinium-functionalized solid phase support will have the structure of formula I, shown above.

These and other aspects of the present invention will become apparent to those skilled in the art after a reading of the following detailed description of the invention, including the illustrative embodiments, examples, and Figures.

### BRIEF DESCRIPTION OF THE DRAWINGS

FIG. 1 is a drawing illustrating the chemistry for attachment of NSP-DMAE to a solid phase resin in the present invention.

FIG. 2 is a drawing illustrating the points at which structures of various NSP-DMAE-pteroate conjugates were varied in the present invention.

FIG. 3 is a drawing illustrating the synthetic sequence of reactions for the synthesis of various NSP-DMAE-spacer-pteroate conjugates of the present invention.

FIG. 4 is a drawing illustrating the synthetic sequence of reactions for the synthesis of various unnatural NSP-DMAE-TEG-folate conjugates of the present invention.

FIG. 5 is a drawing illustrating the synthetic strategy for the solid phase synthesis of DMAE derivatives.

FIG. 6 is a drawing illustrating the decarboxylation of NCM-DMAE-ED.

FIG. 7 is a drawing illustrating the synthetic sequence of reactions for the solid phase synthesis of DMAE-ED-6-CMO-Estradiol conjugate.

FIG. 8 is a drawing illustrating the synthetic sequence of reactions for the solid phase synthesis of DMAE-ED-Theophylline conjugate.

FIG. 9 is a drawing illustrating the synthetic sequence of reactions for the solid phase synthesis of DMAE-ED-Pteroate conjugate.

### DETAILED DESCRIPTION OF THE INVENTION

As used herein all terms have their ordinary meaning in the art unless explicitly defined.

The present invention is founded on the discovery that the acridinium nucleus can be reversibly bound to a solid phase support to provide an acridinium-functionalized solid phase support useful in solid phase synthesis of acridinium compounds, including acridinium compound derivatives and conjugates. The term “acridinium compound” is intended to include any molecule comprising the “acridinium nucleus” shown below:

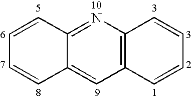

The ring numbering system shown in the acridinium nucleus above is used throughout this disclosure. The term “acridinium compound derivative” is intended to include compounds having substituents at any position on the acridinium nucleus. The term “acridinium compound conjugate” refers to any acridinium compound which is linked to another molecule such as a biologically active molecule, including without limitation, steroids, vitamins, hormones, therapeutic drugs, peptides, nucleic acids, and the like.

In the broadest embodiment, the present invention provides an acridinium-functionalized solid-phase support comprising a solid phase support having immobilized thereon an acridinium compound, preferably a chemiluminescent acridinium compound. The chemiluminescent acridinium compound comprises a linker group covalently attached to the nitrogen atom of the acridinium nucleus (position 10) and the solid phase support. There is essentially no limitation on the nature of the linker. Typically, the linker will be a moiety which permits the cleavage of the acridinium compound from the solid phase support under a given set of conditions, such as acid or base hydrolysis, nucleophilic displacement, or the like. The linker will comprise a functional group, which is capable of bonding with a group on the solid phase support. Preferably the linker is a traceless linker. As used herein, the term “traceless” linker refers to a linker that is an intrinsic part of the acridinium compound structure which can be attached to a solid phase support and which retains its original structure after cleavage from the solid phase.

Suitable functional groups on the linker for reversible traceless attachment to the solid phase support include nucleophiles and electrophiles, such as, for example, hydroxyls, sulfhydryls, carboxyls, sulfonates, and the like.

Preferred traceless linkers comprise carboxyl (—CO₂H) or sulfonyl groups (—SO₃H). These functional groups are capable of reacting with nucleophilic groups, such as hydroxyl, on the surface of the solid phase support to form covalent bonds. In the specific case of reaction with hydroxyl groups on the solid phase support, carboxylate esters and sulfonate esters are formed, respectively, which can be cleaved under acid or base hydrolysis.

Preferred linker moieties are defined by the structures —(CH₂)ₙ—CO₂H and —(CH₂)ₙ—SO₃H, or salts thereof, where n is an integer between 1 and 10, and more preferably n is 1 to 4. It will be understood that, throughout this disclosure, the left-hand side of the linker structure represents the end which is bonded to the ring nitrogen of an acridinium nucleus and the right-hand side represents the end which is bonded to or is capable of binding to the solid phase support. It is contemplated that in some embodiments, the alkyl chain of the linker may be branched or straight chain, optionally comprising one or more unsaturated bonds, and optionally comprising one or more heteroatoms.

In one interesting embodiment, the acridinium-functionalized solid-phase support has the structure of formula I:

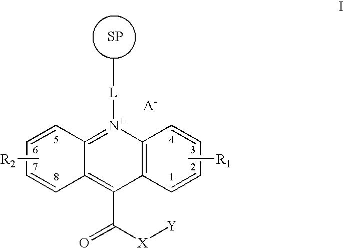

In preferred embodiments of formula I, L is a sulfonate ester or carboxylate ester linker group between the nitrogen of the acridinium nucleus and the solid phase support. Preferably, L represents —(CH₂)ₙ—(C═O)—O— or —(CH₂)ₙ—S(═O)₂—O— where n is an integer between 1 and 10, and more preferably n is 1, 2, 3 to 4.

In formula I, R₁ represents a substituent at one or more of carbon atoms 1-4 and R₂ represents a substituent at one or more of carbon atoms 5-8. R₁ and R₂ are independently selected at each occurrence from the group consisting of hydrogen, substituted or unsubstituted alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof.

In formula I, X represents O, S, or NR^{a}; where R^{a} is —SO₂—R′, R′ being selected from the group consisting of, substituted or unsubstituted, branched or straight chain alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof. In preferred embodiments, X is oxygen.

In the case where X is O or S, Y is a substituent of the formula:

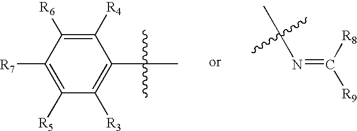

wherein at least one of R₃, R₄, R₅, R₆, R₇, R₈, and R₉ is independently a group -Q-R₁₀, wherein R₁₀ is a group comprising one or more reactive functional groups; where Q represents a bond or a functional group selected from the group consisting of branched or straight-chain alkyl, alkenyl, alkynyl, substituted or unsubstituted aryl, alkyl-aryl, and aryl-alkyl, optionally containing one or more heteroatoms selected from the group consisting of oxygen, nitrogen, phosphorous, sulfur, halogen, and combinations thereof;

and wherein any of R₃, R₄, R₅, R₆, R₇, R₈, and R₉ which are not a group -Q-R₁₀ are substituents independently selected from the group consisting of substituents defined above for R₁ and R₂, hydroxyl, halogen, alkyl, alkenyl, alkynyl, aryl, alkyl-aryl, or aryl-alkyl, —OR^{b}, —SR^{b}, cyano, carboxyl, —(C═O)—OR^{b}, —NR^{b}R^{c}, or —(C═O)—NR^{b}R^{c}, where R^{b} and R^{c} are independently selected from the substituents defined above for R₁ and R₂.

In the case of formula I where X is NR^{a}, Y is a group -Q-R₁₀ as defined above.

The substituent R₁₀ will comprise a reactive functional group, which provides a site for chemical elaboration of the acridinium compound to form acridinium compound derivatives or conjugates. In one embodiment, R₁₀ will comprise one or more nucleophilic groups, electrophilic groups, and combinations thereof.

Suitable nucleophilic groups include, without limitation, nucleophiles selected from the group consisting of amino, hydroxyl, sulfhydryl, sodium or lithium organometallic moieties, or an active methylene group adjacent to a strong electron-withdrawing group; such electron-withdrawing groups consisting of —NO₂, —CN, —SO₂OR*, —N(R*)₃⁺, —S(R*)₂⁺, and —COOR*, wherein R* is selected from the group consisting of, substituted or unsubstituted, branched or straight chain alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof.

In another embodiment, R₁₀ will comprise one or more electrophilic groups. Preferred R₁₀ substituents according to this embodiment are selected from the group consisting of:

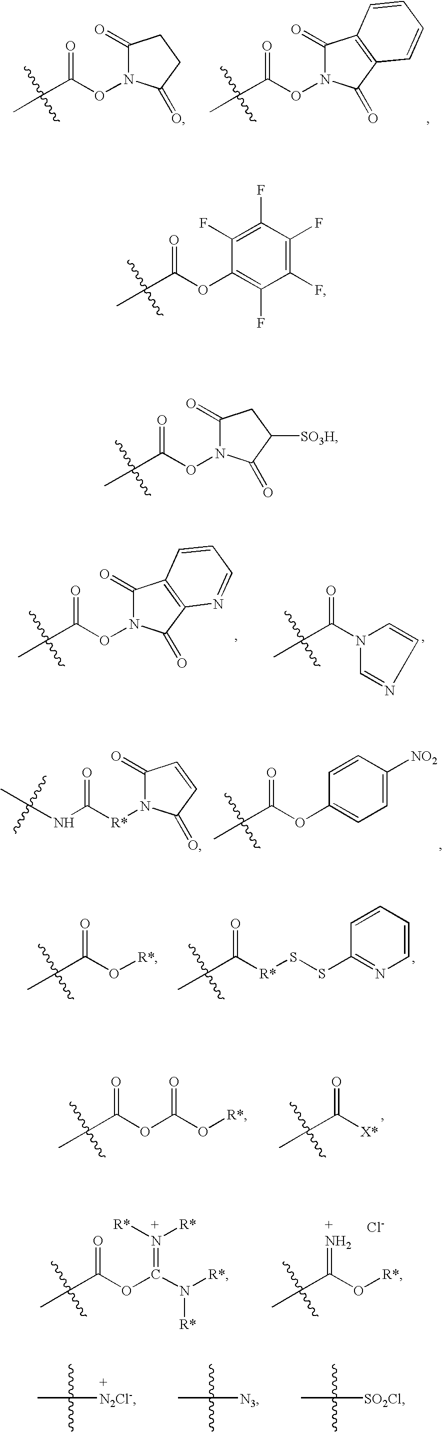

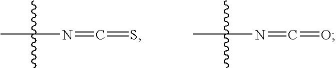

wherein X* is a halogen; and R* is a functional group selected from the group consisting of, substituted or unsubstituted, branched or straight chain alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof.

The identity of Q is not particularly limited. When present, Q will typically, although not necessarily, comprise a carbonyl moiety through which it is attached to the substituent Y. Exemplary functional units which Q may comprise as a point of attachment to Y include those shown below:

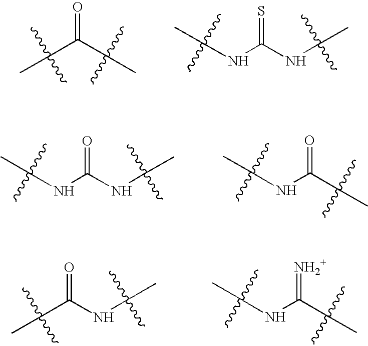

In one interesting embodiment, Q represents the group —(C═O)—NH—R₁₁—NH—R₁₂—, where R₁₁ and R₁₂ are independently selected from the group consisting of alkyl, alkenyl, alkynyl, aryl, aryl-alkyl, and alkyl-aryl, optionally containing one or more heteroatoms selected from the group consisting of oxygen, nitrogen, phosphorous, sulfur, halogen, and combinations thereof. R₁₁ may be, for example, a polyethylene oxide. One exemplary Q group according to this embodiment is —NH—(CH₂CH₂O)ₙ—CH₂CH₂NH—C(O)(CH₂)ₘ— where n=0-20 and m=1-4. In one interesting embodiment, —NH—R₁₁—NH—will be the following structure:

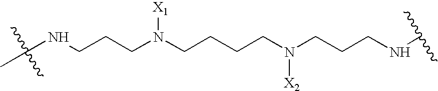

where X₁ and X₂ are independently selected from the following groups:

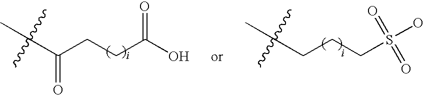

where i is 1 or 2.

A⁻ represents a counter ion. The identity of the counter ion is not of particular importance. In preferred embodiments, the counter ion A⁻ is selected from the group consisting of CH₃SO₄⁻, FSO₃⁻, CF₃SO₄⁻, C₄F₉SO₄⁻, CH₃C₆H₄SO₃⁻, halide, CF₃COO⁻, CH₃COO⁻, NO₃⁻, and combinations thereof.

SP represents a solid phase support. There is essentially no restriction on the nature of the solid phase support other than the requirement that it be functionalized in a manner which permits a bond to be formed with the linker, and preferably in a reversible manner, and more preferably, in a traceless manner. Preferred solid-phase supports have hydroxyl functional groups available to form carboxyl esters or sulfonate esters with the preferred linkers. Preferred solid phase supports include without limitation polystyrene Wang resin, paramagnetic particles, latex particles (including magnetic latex particles), and microtiter plates. It will be understood the circle surrounding the abbreviation SP used in the structures herein is merely for the sake of illustration and is not intended to limited the solid phase support to a spherical structure. The solid phase support may be any structure, including without limitation, beads, amorphous structures, flat surfaces, and the like.

In one currently preferred embodiment of the invention, the acridinium-functionalized solid phase support has the following structure:

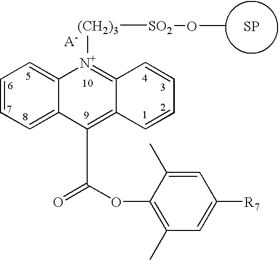

wherein A⁻, R₇ and SP are as defined as above.

In the especially interesting case where R₇ is perfluorophenoxylcarbonyl, the acridinium-functionalized solid phase support will have the following structure:

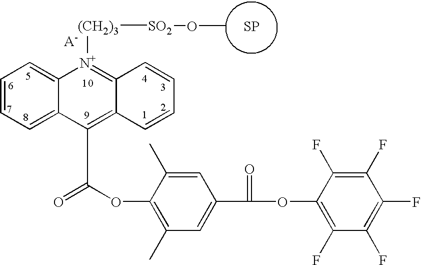

wherein A⁻ and SP are as defined as above.

Another currently preferred acridinium-functionalized solid-phase support has the structure:

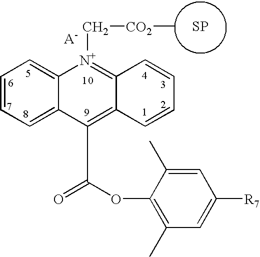

wherein A⁻, R₇ and SP are as defined as above. The perfluorophenoxylcarbonyl shown below is also a currently preferred embodiment:

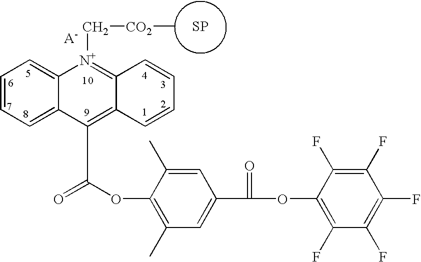

wherein SP and A⁻ are defined as above.

Other suitable acridinium compounds for use in the present invention are described in U.S. Pat. Nos. 6,355,803, 6,664,043, 6,673,569, and 6,783,948, the disclosures of which are hereby incorporated by reference.

The synthetic methodology of the invention is applicable to a wide variety of acridinium compounds. In the preferred practice of the inventive synthetic methodology, the solid phase synthesis of NSP-DMAE and DMAE derivatives is provided. By extension, this methodology is also applicable to other classes of acridinium compounds such as acridinium sulfonamides. The methodology of the present invention is useful for the rapid construction of acridinium compound conjugates of small molecule analytes, which can then be screened for optimal assay performance. The methodology of the present invention also enables the use of chemiluminescent acridinium compounds, emitting light at different wavelengths, to be used for de-convolution of complex libraries or mixtures of compounds generated by combinatorial chemistry.

The methodology of the present invention entails attachment of the acridinium compound to a solid phase using a functional group located on the acridinium nitrogen and; using a second functional group located either on the phenol or sulfonamide leaving group or alternatively at other positions on the acridinium ring, for synthetic elaboration to form a new acridinium compound derivative, followed by cleavage of the acridinium compound derivative from the resin. More specifically, the preferred method entails attachment of acridinium esters containing functional groups on the acridinium nitrogen to solid phases; using a second functional group located either on the phenol leaving group or at other positions in the acridinium ring for synthetic elaboration to form a new acridinium ester derivative followed by cleavage of the acridinium ester derivative from the resin. The functional groups on the acridinium nitrogen that are useful for attachment to a solid phase are N-sulfoalkyl groups, preferably N-sulfopropyl (NSP) or N-sulfobutyl (NSB) groups and, N-carboxymethyl groups (NCM). The functional groups on the phenol or acridinium ring that can used for synthetic elaboration are numerous and examples of which can be found in any standard textbook in organic chemistry such as Smith et al., Advanced Organic Chemistry: Reactions, Mechanisms and Structure (5^{th} Edition Wiley-Interscience). By synthetic elaboration it is meant that the functional group is transformed by reactions to other functional groups or molecules.

The solid phases or resins that are useful are typically, although not necessarily, made of cross-linked polystyrene and contain various functional groups on their surfaces for the attachment of molecules with different functional groups. A number of such solid phases or resins are available from commercial vendors such as Advanced Chemtech Inc. One type of solid phase or resin that is commonly used in solid phase synthesis and which is preferred in the practice of the present invention is the Wang resin which is well known in the art and disclosed in, for example, S. S. Wang J. Am. Chem. Soc. vol 95, p 13128, (1973), the disclosure of which is hereby incorporated by reference. Polystyrene Wang resin ([4-(Hydroxymethyl)phenoxymethyl]polystyrene) is commercially available from numerous vendors, including for example, Aldrich. Polystyrene Wang resin is made of polystyrene and contains benzyl alcohol functional groups on the surface of the resin for the attachment of molecules containing carboxylic acids or sulfonic acids. While the polystyrene Wang resin (PS-Wang) is a preferred resin of this type, it will be understood that other Wang-type resins can also be used. Other solid phases include paramagnetic particles and latex particles, including magnetic latex particles. The advantage of using these particles in solid phase synthesis is the facile separation of the reagents from the particle by magnetic separation. Yet another useful solid phase in the present application is a microtiter plate, which is widely used in solid phase synthesis.

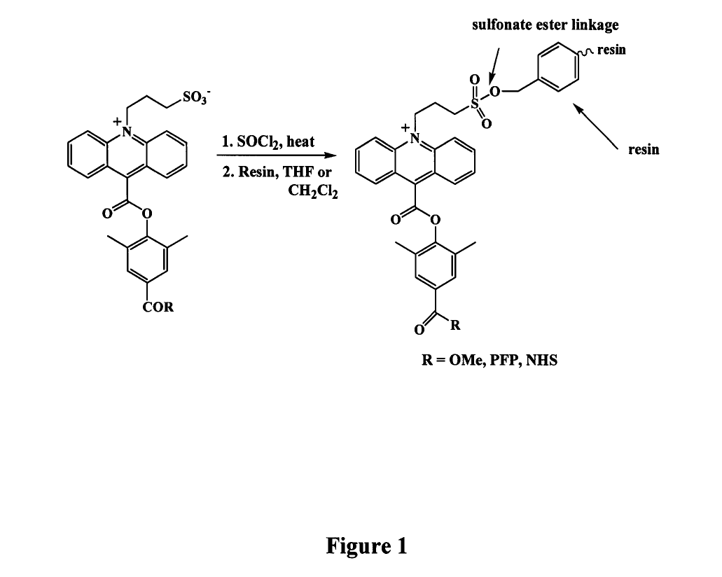

In one interesting embodiment, the present invention provides a method for the solid phase synthesis of NSP-DMAE and DMAE derivatives on polystyrene Wang resin. Advantageously, the methodology of the present invention for the attachment and cleavage of the NSP-DMAE derivative does not entail modification of the acridinium ester with additional functional groups to permit its attachment to the solid phase. By utilizing the sulfonate moiety which, is an inherent part of the structure of NSP-DMAE as a handle for attachment and cleavage from the Wang resin, a ‘traceless linker’ approach for the solid phase synthesis of such compounds and their derivatives is achieved, as illustrated in FIG. 1. FIG. 1 shows the attachment of NSP-DMAE derivatives to Wang resin through conversion of the sulfonate moiety in NSP-DMAE derivatives to the sulfonyl chloride followed by coupling to Wang resins (see Table 1). Conversion of the sulfonate in NSP-DMAE methyl ester, NSP-DMAE-PFP (PFP=pentafluorophenyl) ester and NSP-DMAE-NHS (NHS=N-hydroxysuccinimide) ester to the sulfonyl chloride is accomplished by heating the compounds in neat thionyl chloride as shown in FIG. 1. Covalent attachment of the NSP-DMAE derivative is then accomplished by reacting the sulfonyl chloride with the alcohol groups on the resin in a solvent such as dichloromethane or tetrahydrofuran to from a benzylic sulfonate ester linkage. These chemical transformations are standard techniques in synthetic organic chemistry and are well known to practitioners in the field. Cleavage of resin-immobilized NSP-DMAE is also easily accomplished with acid treatment. Thus, treating the resin (with immobilized NSP-DMAE derivative) with trifluoroacetic acid in dichloromethane, hydrolyzed the sulfonate ester and released the acridinium ester into solution. The solution containing the acridinium ester can then be separated from the resin by a simple filtration step. The extent of acridinium ester immobilization and cleavage can be determined by UV-Visible spectrophotometric analysis. The acridinium ring exhibits a strong absorption band at 370 nm in acid solution such as a 1:1 mixture of water and acetonitrile, each containing 0.05% trifluoroacetic acid. By using this protocol, it was observed that various NSP-DMAE derivatives could be efficiently coupled and cleaved from polystyrene Wang resin (Table 1).

TABLE 1
Attachment and Cleavage of NSP-DMAE
Derivatives to PS-Wang Resin

| | Thionyl chloride, | Immobiliza- | HPLC purity of |
|---|---|---|---|
| | reaction tempera- | tion + cleavage | cleaved NSP-DMAE |
| R | ture, time (h) | efficiency, % | Derivatives, % |
| —OMe | 80° C., 1-2 | 48 | 98 |
| PFP | 50-60° C., 3 | 35 | 99 |
| —O-succi- | 50-60° C., 4 | 36 | 90 |
| nimidyl | | | |

From Table 1, the purity of the cleaved NSP-DMAE derivative as determined by HPLC (High Pressure Liquid Chromatography) was very high, thereby demonstrating that the functional groups on the phenol (methyl ester, pentafluorophenyl ester and NHS ester) were not compromised by the sequence of reactions involved in the conversion of the NSP-DMAE derivative to the sulfonyl chloride, its attachment to the resin and its subsequent cleavage from the resin. Moreover, it was observed that following immobilization of the NSP-DMAE derivative on the resin, the resin could be filtered off and the solvent containing the residual acridinium ester (not attached to the resin) could be recycled. It is apparent from the above, that acridinium compounds functionalized with sulfonate functional groups at other positions on the acridinium ring can be also be covalently coupled to and cleaved from Wang-type resins using the methodology of the present invention. For example, the acridine nitrogen can be alkylated with commercially available 1,4-butane sultone as described by Natrajan et. al in U.S. Pat. No. 6,355,803, the disclosure of which is hereby incorporated by reference. The N-sulfobutyl group can then be processed as described herein.

In another embodiment, the immobilization and cleavage methodology can also be used for the construction of acridinium conjugates of folic acid analogs. The vitamin folic acid is a conjugate of glutamic acid and pteroic acid and is commonly measured by immunoassays. Folic acid is a small analyte and in automated immunoanalyzers, for example Siemens Medical Solutions Diagnostics' ACS:180® and Advia Centaur®, the folate assay employs an acridinium conjugate of folic acid and, folate binding protein (FBP) immobilized onto paramagnetic particles (PMP) as the two main assay reagents. In folic acid, one of two carboxylic acids (referred to as alpha and gamma) is commonly used for the preparation of conjugates. For efficient binding of the folate conjugate to folate binding protein, the alpha (α) carboxylic acid should be free and the gamma (γ) carboxylic acid should be the preferred site of attachment, as described in Wang et al. Bioconjugate Chem. 1996, vol 7, p 56-62, the disclosure of which is hereby incorporated by reference. The structure of folic acid is shown below.

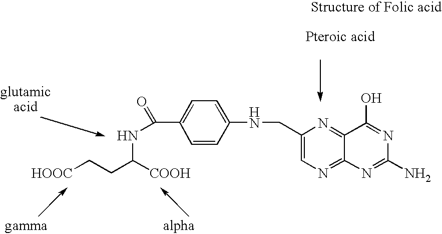

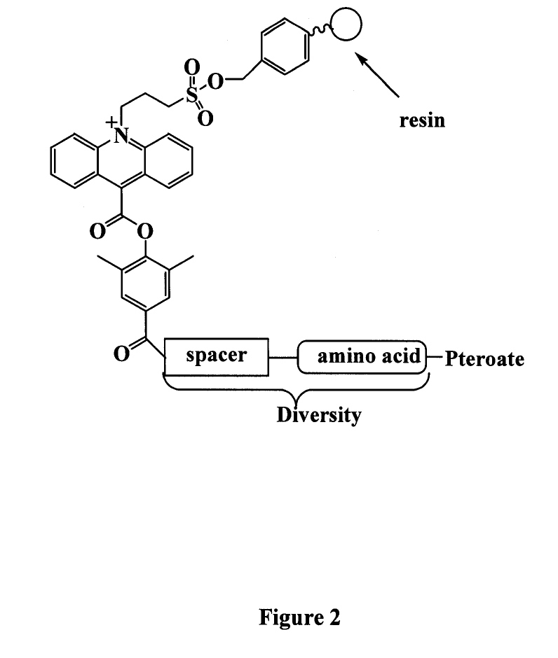

There are two structural aspects of NSP-DMAE-folate conjugates, which are relevant to the binding of these conjugates to FBP and are illustrated in FIG. 2. Normally, during the preparation of chemiluminescent or fluorescent conjugates of small analytes, a ‘spacer’ is introduced between the analyte and chemiluminescent or fluorescent molecule. The spacer or linker serves many functions and, for a given analyte, its structure may require optimization. One function of the spacer is to minimize steric interference of the chemiluminescent or fluorescent label on the binding of the conjugate to its binding molecule. Binding molecules that are commonly used are antibodies or binding proteins in immunoassays and, nucleic acids in nucleic acid assays. The spacer may also influence the solubility of the conjugate and hydrophilic spacers with improved aqueous solubility derived from poly(ethylene) glycol and spermine have been disclosed by Natrajan et. al in U.S. Pat. No. 6,664,043, the disclosure of which is hereby incorporated by reference. These hydrophilic spacers were found to confer beneficial properties such as enhanced specific binding and lower non-specific binding on acridinium ester-analyte conjugates for the analytes folate, theophylline and tobramycin.

The spacer length was optimized for maximal binding of NSP-DMAE-pteroate conjugates by screening several spacers. Even though these conjugates do not contain the glutamic acid moiety that is found in folate, it has been discovered that the pteroate moiety by itself also binds to FBP although less well than the γ-linked folate conjugate. The spacers that were screened included ethylene diamine (ED), and other diamino molecules derived from di(ethylene) glycol (DEG), tri(ethylene) glycol (TEG),

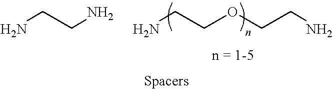

tetra(ethylene) glycol (TEEG), penta(ethylene) glycol (PEEG) and hexa(ethylene) glycol (HEG).

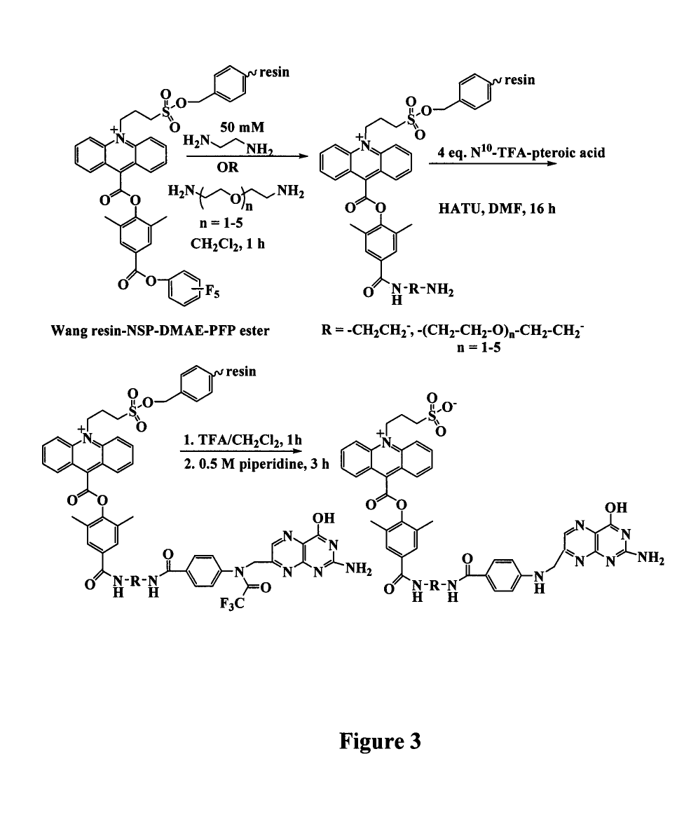

The solid phase synthesis of NSP-DMAE-pteroate conjugates may be accomplished as illustrated in FIG. 3. Polystyrene Wang resin-immobilized NSP-DMAE-PFP ester was first reacted with the above diamino compounds. In these reactions, the PFP ester was replaced with an amide linkage between one of the amines in the spacer leaving the other end free for subsequent reaction with N¹⁰-trifluoroacetyl pteroic acid. This coupling reaction was mediated by the commercially available coupling reagent HATU, which is commonly used in peptide synthesis. In this second reaction an amide bond was formed between resin-immobilized NSP-DMAE-spacer and the pteroate moiety. The various NSP-DMAE-spacer-N¹⁰-trifluoroacetyl-pteroate conjugates were then cleaved off the resin with trifluoroacetic acid and separated from the resin by filtration. The conjugates were all purified by HPLC. Removal of the trifluoroacetyl group in the conjugates was accomplished using the organic base piperidine.

In the reactions described above, unintended cleavage of the NSP-DMAE from the resin was not observed. This is advantageous for the solid phase synthesis of acridinium compound derivatives. Normally, the benzyl sulfonate esters are quite susceptible to hydrolysis but the enhanced stability observed with NSP-DMAE attached to Wang resin is attributable to the solid phase. Similar observations with other benzyl sulfonates immobilized on Wang resin were reported by Hari and Miller Org. Lett. 1999, vol 1, p 2109-2111, the disclosure of which is hereby incorporated by reference.

To study the impact of the various spacers in the NSP-DMAE-pteroate conjugates on the binding of the conjugates to FBP, these conjugates were screened in Siemens Medical Solutions Diagnostics' Folate assay. NSP-DMAE-pteroate conjugates with various spacers and NSP-DMAE-folate conjugates with different linkage sites were tested in a folate binding assay (Siemens Medical Solutions Diagnostics' ACS:180® Folate Assay) for comparison of their binding to folate binding protein. For this assay, the above-mentioned conjugates were each diluted to a concentration of 10 nanomoles/L in a solution containing 0.0060 M sodium dihydrogen phosphate, 0.024 M potassium monohydrogen phosphate, 0.015 M sodium azide, 0.15 M sodium chloride, 1.0 g/L bovine serum albumin, at pH 7.4. The ACS:180® Folate Assay is one of a series of commercially marketed immunoassays manufactured by Siemens Medical SolutionsDiagnostics for application on the Siemens Medical SolutionsDiagnostics' ACS:180® (Automated Chemiluminescent Immunoassay System). In this assay, acridinium compound conjugates of folate and pteroate bind to folate binding protein which is immobilized onto to magnetically separable paramagnetic particles (PMP). As acridinium compounds are chemiluminescent and emit light which is measured by the ACS:180® and since folate and pteroate compounds bind to folate binding protein, then a positive correlation exists between the amount of NSP-DMAE-pteroate or -folate conjugate bound to folate binding protein on PMP and the amount of chemiluminescence measured by the ACS:180®. The ACS:180® automatically performed the following steps for the Folate Assay. First, 0.100 mL of each 10 nanomole/L solution of NSP-DMAE-pteroate or -folate conjugate was mixed with 0.480 mL of folate binding protein on PMP for 2.5 minutes at 37° C. The ACS:180® finishes the ACS:180® Folate Assay by magnetically separating the PMP from the fluid containing unbound NSP-DMAE-pteroate conjugate or -folate conjugate and then washes the PMP with water. Chemiluminescence from acridinium compound on the PMP was initiated with subsequent light emission with sequential additions of 0.30 mL each of ACS:180® Reagent 1 and Reagent 2. Reagent 1 was 0.1 M nitric acid and 0.5% hydrogen peroxide. Reagent 2 was 0.25 M sodium hydroxide and 0.05% cetyltrimethylammonium chloride. The ACS:180® measured the chemiluminescence corresponding to each NSP-DMAE-pteroate or -folate conjugate that was tested as relative light units (RLUs). The amount of chemiluminescence measured is correlated to the amount of NSP-DMAE-pteroate or -folate conjugate that will bind to the PMP; consequently, the amount of chemiluminescence is correlated to the affinity of an NSP-DMAE-pteroate or -folate conjugate for folate binding protein, as the amount of each tested NSPDMAE-pteroate or -folate conjugate was the same, meaning that the greater the affinity a NSP-DMAE-pteroate or -folate conjugate for folate binding protein then the greater the number of RLUs that were measured.

Normalization to percentage of chemiluminescence measured compared to the total chemiluminescence in 0.100 mL of 10 nanomoles of NSP-DMAE-pteroate or -folate conjugate yields tabulated relative affinities (Bo/T) of NSP-DMAE-pteroate or -folate conjugates with various spacers or sites of linkage for folate binding protein. The higher the percentage of bound chemiluminescence measured for a NSP-DMAE-pteroate or -folate conjugate then the greater the affinity of that conjugate for folate binding protein.

The results are presented in tabular form in Table 2 for these various NSP-DMAE conjugates of pteroic acid, NSP-DMAE-ED-Pteroate, NSP-DMAE-DEG-Pteroate, NSP-DMAE-TEG-Pteroate, NSP-DMAE-TEEG-Pteroate, NSP-DMAE-PEEG-Pteroate and NSP-DMAE-HEG-Pteroate.

TABLE 2
Binding of NSP-DMAE-Pteroates to FBP

| Compound | Bo/T, % |
|---|---|
| NSP-DMAE-ED-Pteroate | 0.82 |
| NSP-DMAE-DEG-Pteroate | 0.92 |
| NSP-DMAE-TEG-Pteroate | 1.26 |
| NSP-DMAE-TEEG-Pteroate | 1.13 |
| NSP-DMAE-PEEG-Pteroate | 1.15 |
| NSP-DMAE-HEG-Pteroate | 1.10 |

From Table 2, it is seen that the binding of the conjugates increases with spacer length until an optimal length is reached which, corresponds to the TEG spacer. Further increase in spacer length in the conjugates containing the TEEG, PEEG and HEG spacers, does not improve the binding any further. Thus, spacer length for optimal binding could be determined rapidly by this simple study entailing the rapid assembly of NSP-DMAE-Pteroate conjugates with different length spacers on a solid phase and screening them for binding in the Siemens Medical Solutions Diagnostics' folate assay.

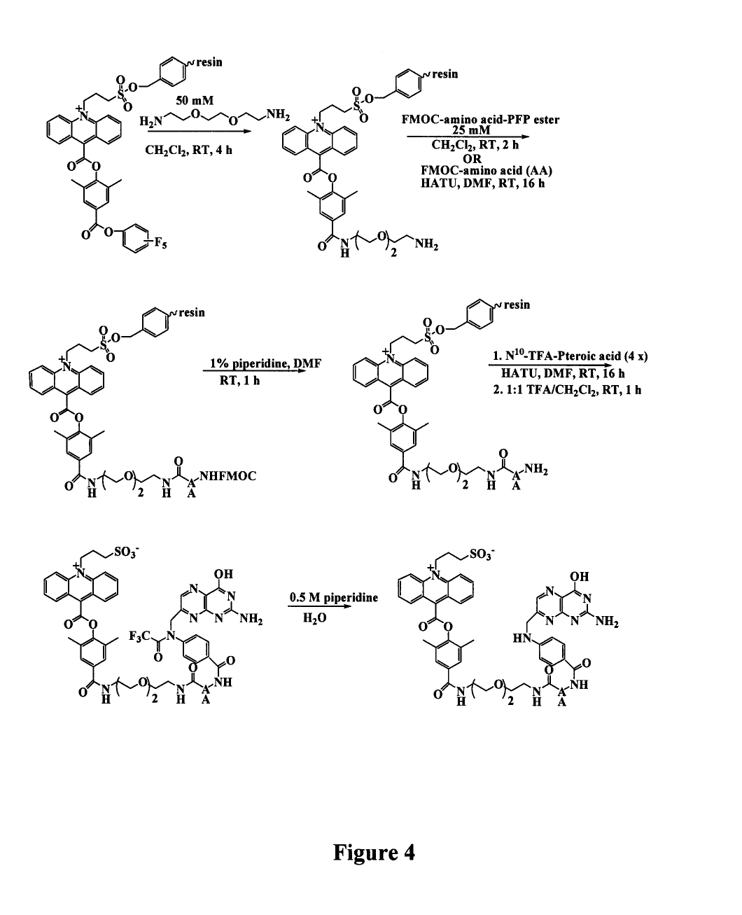

With the length of the spacer optimized, the synthetic methodology of the present invention was used to investigate the effect of various amino acid substitutions in ‘unnatural’ folate conjugates of NSP-DMAE with the TEG spacer. The folate conjugates are considered unnatural because the amino acid glutamic acid which, is normally found in folate, has been replaced with other amino acids in these conjugates, such as, for example, the amino acids alanine (Ala), arginine (Arg), glutamine (Gln), histidine (His), isoleucine (Isoleu), leucine (Leu), methionine (Met), norleucine (Norleu), phenylalanine (Phe), proline (Pro), serine (Ser), tyrosine (Tyr), and valine (Val). The solid phase syntheses of these conjugates was accomplished as illustrated in FIG. 4. All the chemical reactions and reagents depicted in FIG. 4 are well known to practitioners in the field of synthetic organic chemistry.

The solid phase synthesis was begun with 0.9 g of polystyrene Wang resin with NSP-DMAE-PFP ester attached to resin by a sulfonate ester linkage from the sulfonate moiety of the acridinium ester to the benzyl alcohols on the resin. Reaction of the PFP ester with diamino-TEG displaced the PFP ester and afforded resin-immobilized NSP-DMAE-TEG. A small portion of the resin was subjected to treatment with trifluoroacetic acid in dichloromethane to cleave the acridinium ester from the resin. Subsequent analysis by HPLC indicated that the product NSP-DMAE-TEG had a purity of 94% thereby indicating clean formation of this NSP-DMAE compound on the Wang resin. Analysis of acridinium ester yield by UV-Visible spectrophotometry, as discussed earlier, also indicated an overall yield of 85%. Thus, these results indicate that the reaction of resin-immobilized NSP-DMAE-PFP ester with diamino-TEG proceeded to completion and very little acridinium ester was cleaved off the resin during the reaction. Resin-immobilized NSP-DMAE-TEG (25 mg resin per reaction) was then coupled to various FMOC-protected (FMOC=fluorenylmethyl)amino acids either using the coupling reagent HATU, which is used commonly in peptide synthesis or, by direct reaction with FMOC-amino acid-PFP esters. The FMOC protecting group is commonly used in peptide syntheses and FMOC-protected amino acids are wellknown in the art. Following these coupling reactions, resin-immobilized NSP-DMAE-TEG-FMOC-amino acid in each case was treated with 1% piperidine, which cleaved the FMOC group from the amino acids. To determine whether this treatment also caused any unintended cleavage of the acridinium ester from the resin, for the amino acid phenylalanine, a small portion of the resin following piperidine treatment was treated with trifluoroacetic acid to cleave the NSP-DMAE derivative off the resin. Quantification of the acridinium ester from this treatment indicated a yield of 100%. Thus, piperidine treatment did not compromise the sulfonate ester linkage from NSP-DMAE to the resin.

The final phase of the synthesis was accomplished by coupling N¹⁰-trifluoroacetyl acid to resin-immobilized NSP-DMAE-TEG-amino acid using the peptide coupling reagent HATU. The various NSP-DMAE-TEG-amino acid-N¹⁰-trifluoracetyl-pteroate conjugates were then cleaved from the resin using trifluoroacetic acid and purified by HPLC. Overall acridinium ester yields were measured for the amino acids phenylalanine and proline and were observed to be 81% and 72%. The final reaction in all the conjugates to cleave the trifluoroacetyl group was carried out using aqueous piperidine. The structures of the various unnatural NSP-DMAE-folate conjugates are shown below.

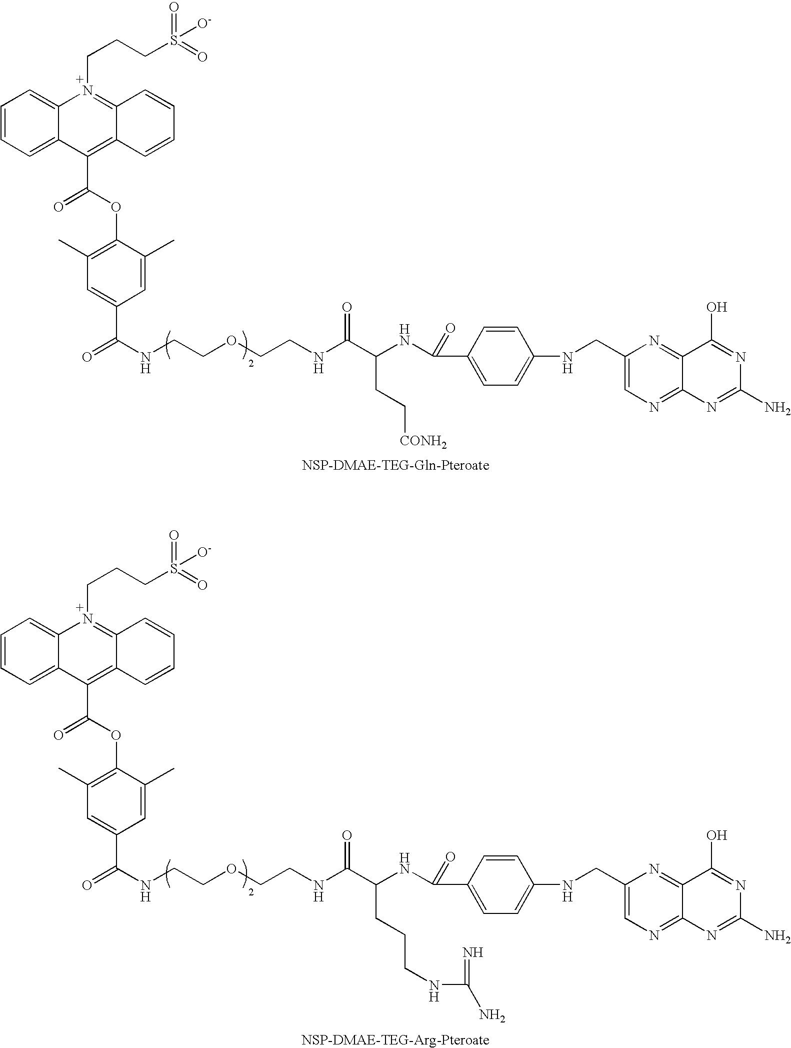

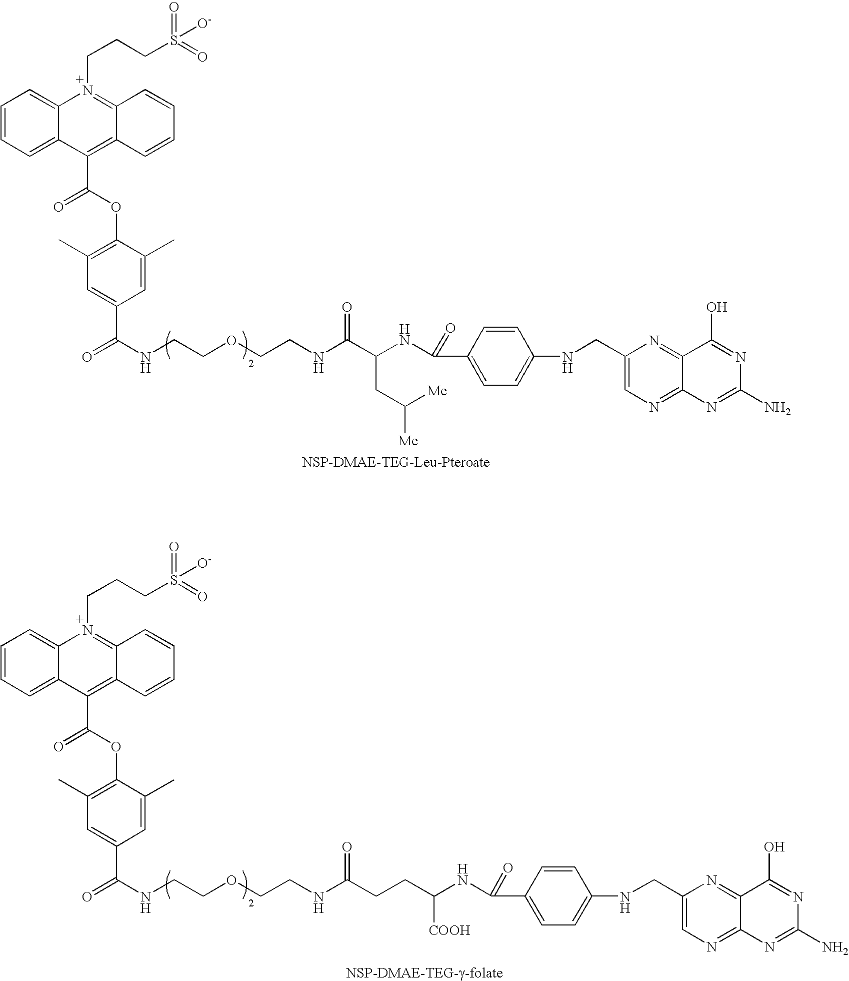

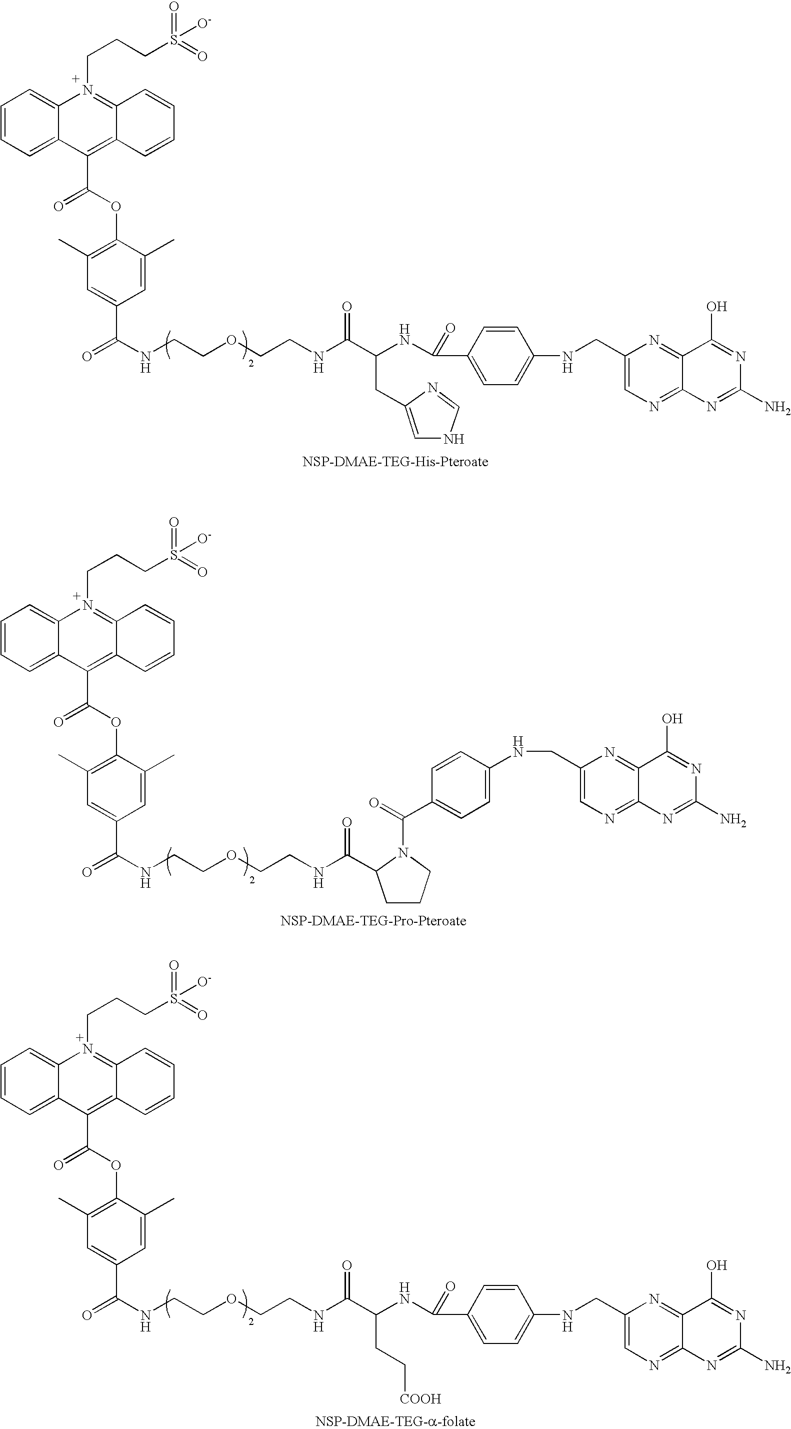

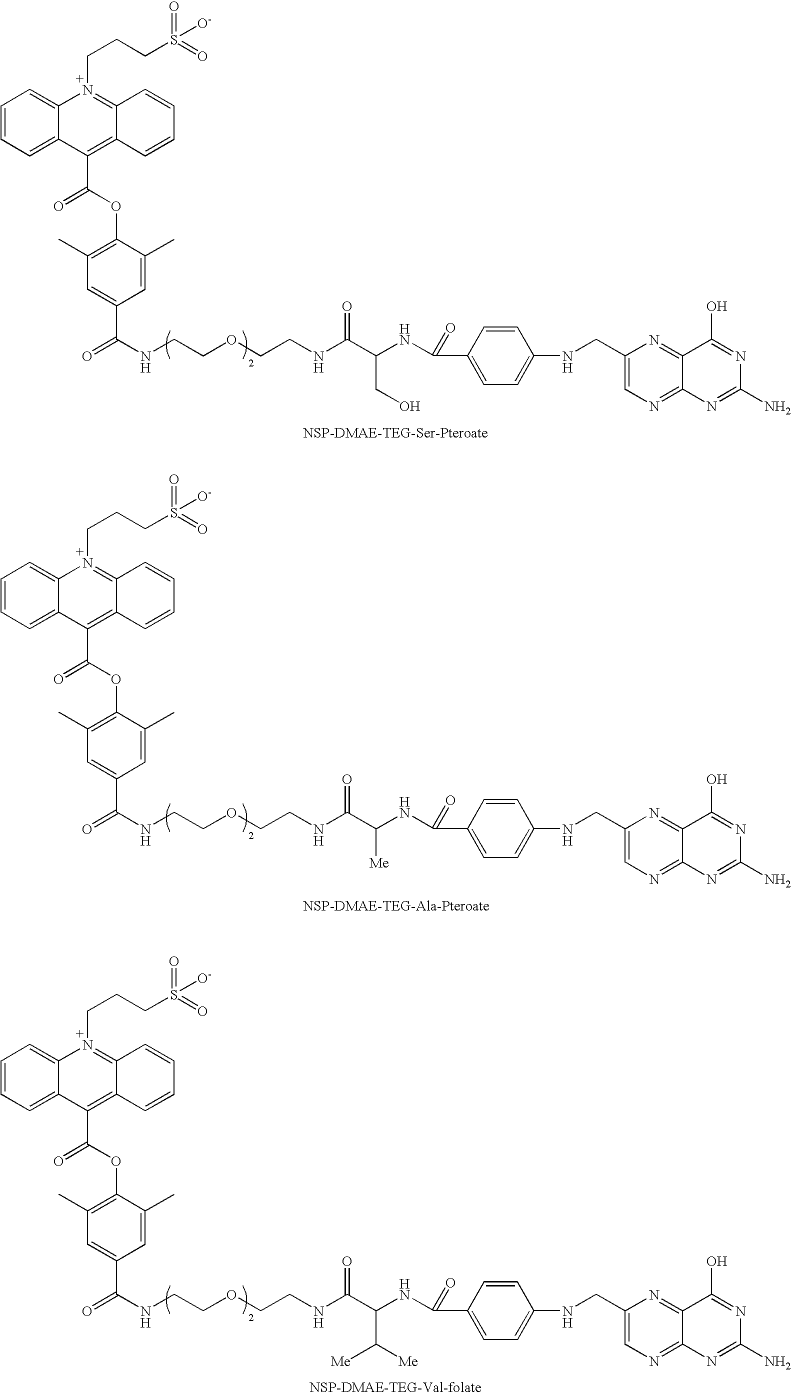

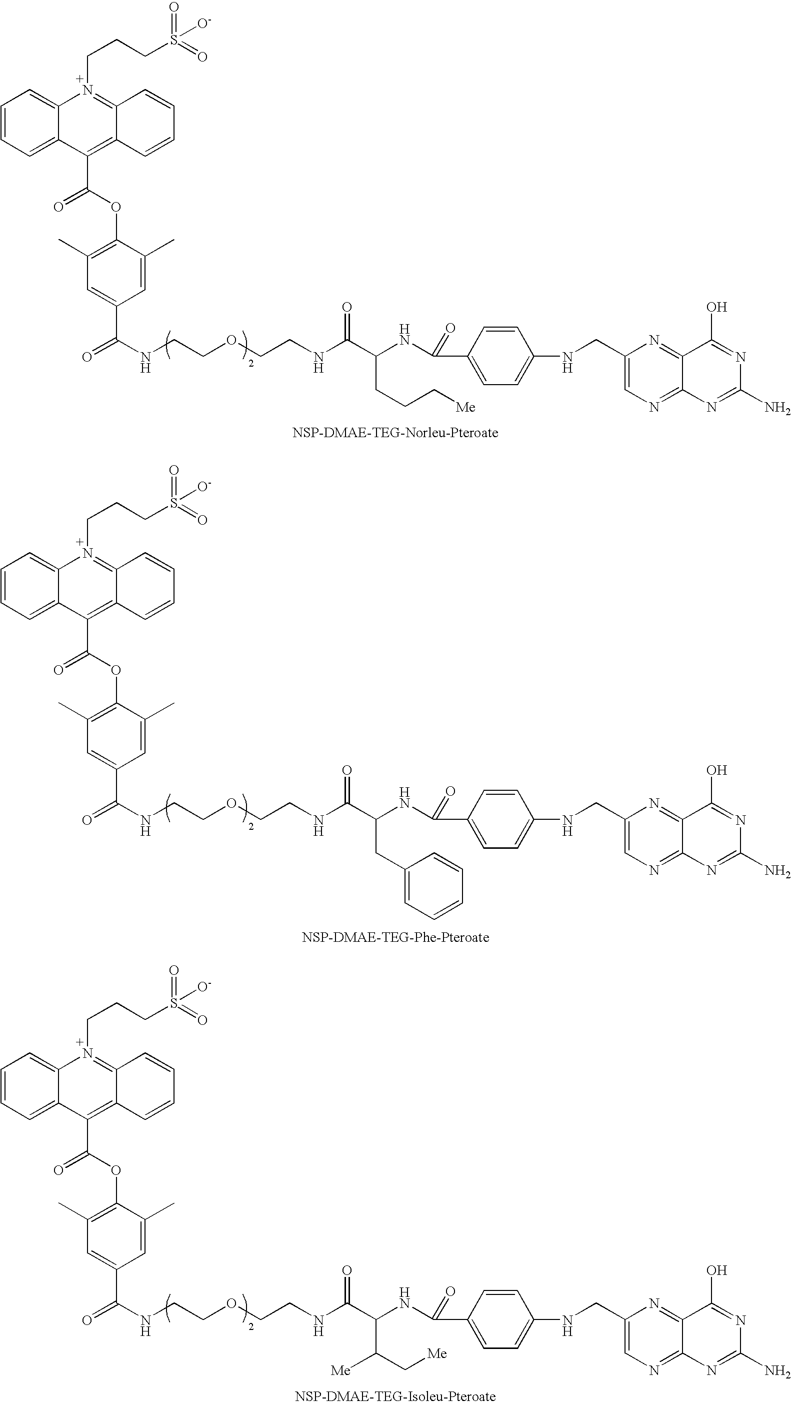

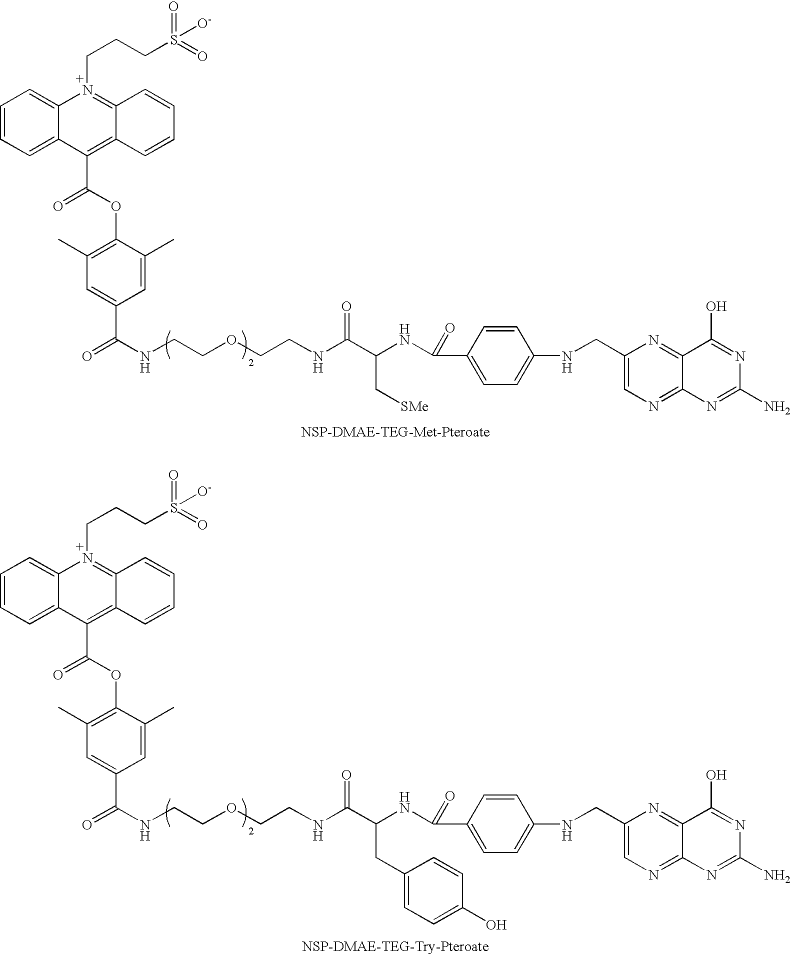

The binding of the various unnatural folate conjugates to folate binding protein (FBP) was evaluated next in the Siemens Medical Solutions Diagnostics folate assay as described earlier for the NSP-DMAE-Pteroate conjugates. The results are presented in Table 3. Also included in Table 3 are the binding values of two folate conjugates with the natural amino acid glutamic acid. The conjugate NSP-DMAE-TEG-α-folate has a free γ-carboxylic acid whereas the conjugate NSP-DMAE-TEG-γ-folate has a free α-carboxylic acid. These two conjugates were synthesized using the procedures described by Natrajan et al. in U.S. Pat. No. 6,664,043, the disclosure of which is hereby incorporated by reference.

TABLE 3
Binding of unnatural and natural NSP-DMAE-
TEG-folate conjugates to FBP

| Conjugate | Bo/T, % | HPLC purity, % |
|---|---|---|
| NSP-DMAE-TEG-Arg-Pteroate | 0.85 | 85 |
| NSP-DMAE-TEG-His-Pteroate | 1.10 | 90 |
| NSP-DMAE-TEG-Gln-Pteroate | 0.95 | 90 |
| NSP-DMAE-TEG-Ser-Pteroate | 0.97 | 90 |
| NSP-DMAE-TEG-Ala-Pteroate | 0.84 | 90 |
| NSP-DMAE-TEG-Pro-Pteroate | 0.76 | 91 |
| NSP-DMAE-TEG-Met-Pteroate | 1.05 | 88 |
| NSP-DMAE-TEG-Val-Pteroate | 0.94 | 94 |
| NSP-DMAE-TEG-Tyr-Pteroate | 1.19 | 96 |
| NSP-DMAE-TEG-Isoleu-Pteroate | 0.85 | 98 |
| NSP-DMAE-TEG-Leu-Pteroate | 0.88 | 99 |
| NSP-DMAE-TEG-Norleu-Pteroate | 0.98 | 92 |
| NSP-DMAE-TEG-Phe-Pteroate | 1.06 | 93 |
| NSP-DMAE-TEG-α-folate | 1.31 | 97 |
| NSP-DMAE-TEG-γ-folate | 2.31 | 96 |

From the above table, the binding values for all the unnatural folate conjugates are lower than the two natural folate conjugates as would be expected. Consistent with reports in the literature, the conjugate linked through the γ-carboxylic acid, NSP-DMAE-TEG-γ-folate has the highest binding which is almost double (2.31) that of the α-linked isomer NSP-DMAE-TEG-α-folate (1.31). Thus, the α-carboxylic acid seems to be an important contributor to binding. Inspection of Table 3 reveals some interesting details about binding of the unnatural folate conjugates to FBP. The lowest binding is observed for the amino acid proline. Without wishing to be bound by any theory, it is believed that this is probably because in this conjugate, the orientation of the acridinium ester in relation to the pteroate moiety results in some degree of steric interference to binding of the conjugate to FBP. In proline, the amino and carboxylic acid functional groups to which the pteroate and acridinium ester are linked respectively, are oriented approximately 90° to one another. This orientation is likely to enhance the steric interference to binding of the pteroate moiety to FBP by the acridinium ester.

In the conjugate containing the basic amino acid arginine, the binding is again low. This result taken in conjunction with the enhanced binding that is observed with NSP-DMAE-TEG-γ-folate, containing a free α-carboxylic acid, suggests that there may be an unfavorable electrostatic interaction between the basic arginine moiety and a basic functional group in the binding site of FBP.

The results in Table 3 also indicate that the unnatural folate conjugates derived from amino acids with aromatic functional groups bind better than those amino acids with aliphatic functional groups. For example binding of the unnatural folate conjugates with the amino acids His, Tyr and Phe are 1.1, 1.19 and 1.06, respectively. These amino acids contain aromatic functional groups. In contrast, the unnatural folate conjugates with the amino acids Ala, Isoleu and Leu have lower binding of 0.84, 0.85 and 0.88 respectively. These amino acids contain aliphatic alkyl functional groups that do not seem to contribute to binding.

The foregoing description of some of the preferred embodiments illustrates the simplicity and utility of the present invention. By rapidly assembling a number of unnatural folate conjugates of NSP-DMAE by solid phase synthesis and screening them for binding, mechanistic and structural details of their binding to FBP could be deduced in a relatively short time. It will be readily apparent to one skilled in the art that the methodology described above can be utilized for any acridinium compound with a N-sulfopropyl or N-sulfobutyl group which can be utilized for covalent attachment to a solid phase such as Wang resins. The second functional group that is required for synthetic elaboration can be located either on the phenol, as described in the present invention, or at other positions in the acridinium ring. And, a variety of acridinium compound derivatives with different structural features can be synthesized by the acridinum compound immobilized solid phase of the present invention.

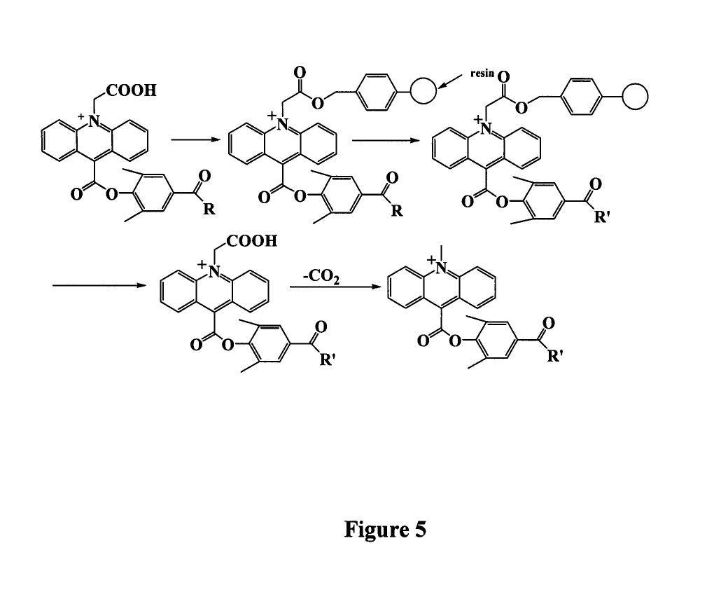

The attachment of acridinium compounds to solid phases such as Wang resins can also be performed using N-carboxymethyl (NCM) groups instead of N-sulfopropyl or N-sulfobutyl groups. In this case, reaction of the N-carboxymethyl group on the acridinium compound and the benzyl alcohol on the Wang resin results in a carboxylate ester bond. Methods for forming ester bonds between carboxylic acids and alcohols are well known in the prior art and constitute standard practices in synthetic organic chemistry. Once the acridinium compound has been immobilized on the Wang resin, then a second functional group on the acridinium compound can be utilized for further synthetic elaboration in a manner similar to what was illustrated for the solid phase immobilized NSP-DMAE derivatives. Cleavage of the carboxylate ester bond from the Wang resin can also be accomplished in the same manner as described earlier for the sulfonate esters, i.e., with treatment with acids such as trifluoroacetic acid. The acridinium compound that is released from the resin by this procedure will contain the N-carboxymethyl group. To convert this acridinium compound to a DMAE derivative, which, contains an N-methyl group, a method to convert to the N-carboxymethyl group to an N-methyl group is needed and is provided by the methodology of the current invention. One synthetic strategy for the solid phase synthesis of DMAE derivatives of the current invention is illustrated in FIG. 5.

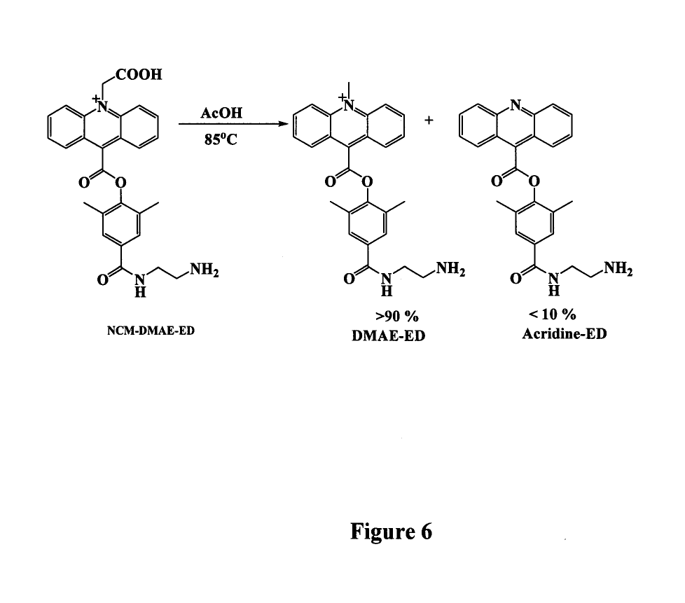

In this approach, a DMAE derivative with an NCM group and containing a functional group R on the phenol, is linked to polystyrene Wang resin to from a carboxylate ester linkage. The functional group R is then used for synthetic elaboration to give the group R′. R′ can be, without limitation, an estradiol, theophylline or pteroate derivative and by analogy, any small molecule. Estradiol and theophylline are small molecule analytes that are commonly measured in immunoassays. The DMAE derivative is then cleaved from the resin and in a final step, the NCM group is converted to the DMAE derivative with an N-methyl group by decarboxylation of the NCM moiety. The decarboxylation of NCM-DMAE derivatives has been heretofore unknown. To demonstrate the feasibility of the synthetic strategy illustrated in FIG. 5, the decarboxylation of NCM-DMAE-ED illustrated in FIG. 6 was investigated. The NCM-DMAE derivative was synthesized using standard organic chemistry techniques and synthetic details are described in Example 4. It was found that the decarboxylation of NCM-DMAE-ED by heating the compound without solvent is accompanied by the undesired loss of the entire N-alkyl group to give the acridine-ED. However, NCM-DMAE derivatives can be decarboxylated efficiently with minimal loss of the N-alkyl group by heating the NCM-DMAE derivative in neat acetic acid. Other reaction conditions were less successful although are contemplated to be within the scope of the invention. These included heating the NCM-DMAE-ED in the solid state either by itself or with salts such as manganese chloride, ammonium chloride or by treatment with acids such as 30% HBr/AcOH and trifluoroacetic acid.

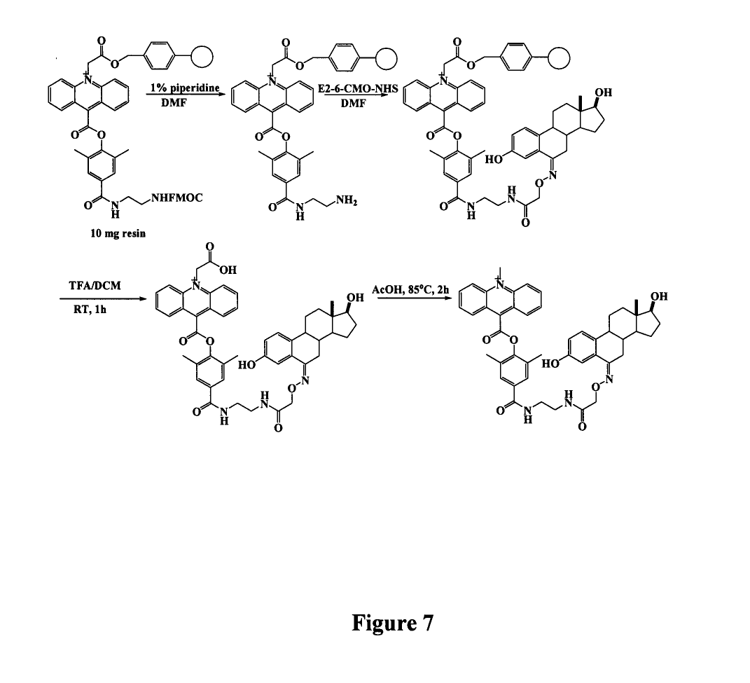

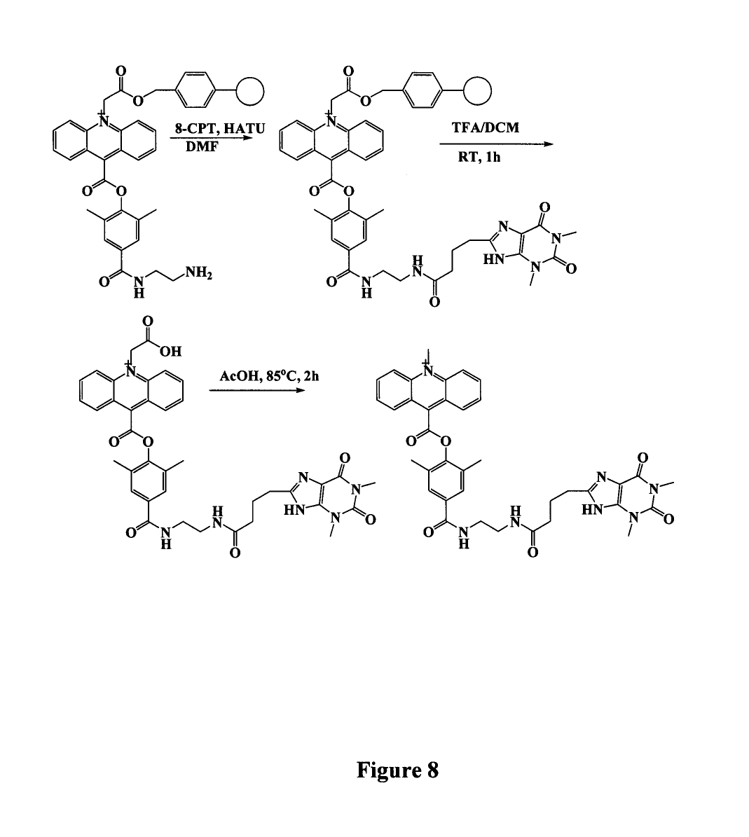

The solid-phase synthesis of three conjugates of DMAE, DMAE-ED-6-CMO-Estradiol, DMAE-ED-theophylline and DMAE-ED-pteroate using the synthetic methodology of the current invention are shown in FIGS. 7-9 respectively and explained in detail in the Examples provided herein. Briefly, the syntheses entailed attachment of NCM-DMAE-ED containing an FMOC protecting group to polystyrene Wang resin; cleavage of the FMOC group; reaction with the estradiol, theophylline or pteroate moieties; cleavage of the DMAE conjugate from the resin followed by decarboxylation by heating the conjugate in neat acetic acid. The estradiol, theophylline and pteroate compounds are available from commercial vendors or can be readily prepared according to well-known literature methods.

Broadly, the methodology for the solid phase synthesis of acridinium compounds and their derivatives or conjugates according to the invention comprises the steps of: (a) attachment of the acridinium compound using a N-sulfoalkyl group to a solid phase, (b) using a second functional group on the acridinium ring or leaving group for synthetic, elaboration to give a new acridinium compound derivative or conjugate, (c) cleaving the acridinium compound derivative or conjugate from the solid phase.

The methodology of the current invention can also be used for the synthesis of acridinium compounds and their conjugates comprising the steps of: (a) attachment of the acridinium compound using a N-carboxymethyl group to a solid phase, (b) using a second functional group on the acridinium ring or leaving group for synthetic elaboration to give a new acridinium compound derivative or conjugate, (c) cleaving the acridinium compound derivative or conjugate from the solid phase, (d) decarboxylating the acridinium compound derivative or conjugate by heating in acetic acid.

### Example 1

### Immobilization of NSP-DMAE onto Polystyrene Wang Resin

### a) Synthesis of 2′,6′-dimethyl-4′-pentafluorophenyloxycarbonylphenyl-10-N-sulfopropyl-acridinium-9-carboxylate (NSP-DMAE-PFP ester)

A solution of 2′,6′-dimethyl-4′carboxyphenyl-10-N-sulfopropyl-acridinium-9-carboxylate (0.105 g, 0.25 mmol) and pentafluorophenol (0.132 g, 0.72 mmol) in anhydrous DMF (10 mL) was treated with diisopropylcarbodiimide (0.115 mL, 3 equivalents). The reaction was stirred at room temperature under nitrogen atmosphere. After 2-3 hours of stirring, HPLC analysis using an analytical C₁₈ column from Phenomenex, 4.6 mm×30 cm and a 30 minute gradient of 10→70% MeCN/H₂O with 0.05% trifluoroacetic acid at a flow rate of 1 mL/min. and UV detection at 260 nm; indicated complete conversion to product eluting at 24 minutes. The reaction mixture was evaporated to dryness and the residue was suspended in toluene and evaporated to dryness. The dried residue was dissolved in methanol and the product was purified by preparative TLC on silica using 20% methanol/chloroform as the eluting solvent. Yield=0.121 g (86%).

### b) Immobilization of NSP-DMAE-PFP Ester onto Polystyrene Wang Resin

NSP-DMAE-PFP ester (0.12 g, 0.21 mmol) was suspended in neat thionyl chloride (1.5 mL) and the suspension was heated in an oil bath at 50° C. for 2.5 hours. The reaction was then concentrated under reduced pressure and the residue was suspended in anhydrous toluene (5 mL) and evaporated to dryness. The acid chloride was then dissolved in anhydrous THF (10 mL) and added to polystyrene Wang resin (1.8-2.0 mmol OH/g, Aldrich, 1 g) along with diisopropylethylamine (0.2 mL). The reaction was stirred gently for 16 hours at room temperature. The reaction was then diluted with methanol (5 mL) and after allowing the resin to settle, the solvent was removed with a Pasteur pipet. The resin was rinsed several times with methanol in this manner. The combined washes were evaporated to dryness to afford unreacted NSP-DMAE-PFP ester, which could be recycled.

The resin, after the final methanol wash, was dried under vacuum. Acridinium ester loading was determined by stirring 10 mg of the resin with 0.5 mL of 1:1, dichloromethane and trifluoroacetic acid for one hour. The reaction was then diluted with methanol (1-2 mL) and filtered to remove the resin. The filtrate was evaporated to dryness. The acridinium ester cleaved from the resin was then dissolved in 1 mL of 1:1, H₂O/MeCN each containing 0.05% trifluoroacetic acid. HPLC analysis of this solution as described in section (a) indicated clean NSP-DMAE-PFP ester of 99% purity. UV-Visible spectrophotometic analysis was performed on a Beckman Model 7500 spectrophotometer. The acridinium ring showed a strong absorption at 370 nm (ε_{M}=10,000). From this analysis the acridinium ester loading on the polystyrene resin was found to be 38 mg per 1 g Wang resin.

### Example 2

### Synthesis of NSP-DMAE-Pteroate Conjugates on Wang Resin

### a) Coupling of Ethylene Diamine (ED), Diamino Di(Ethylene)Glycol (DEG) Diamino tri(ethylene)glycol (TEG), Diamino tetra(ethylene)glycol (TEEG), Diamino Penta(Ethylene)Glycol (PEEG) and Diamino Hexa(Ethylene)Glycol (HEG) to Resin-Immobilized NSP-DMAE-PFP Ester

Wang resin-immobilized NSP-DMAE-PFP ester (20 mg resin, 2 mg acridinium ester) was suspended in dichloromethane (1-2 mL) and treated separately with ethylene diamine (ED), diamino di(ethylene)glycol (DEG), diamino tri(ethylene)glycol (TEG), diamino tetra(ethylene)glycol (TEEG), diamino penta(ethylene)glycol (PEEG) and diamino hexa(ethylene)glycol (HEG) (50-100 mM). The diamino compounds derived from TEEG, PEEG and HEG were synthesized from the commercially available diols as described in U.S. Pat. No. 6,664,043. The reactions were stirred at room temperature for 3.5 hours and were then diluted with methanol (˜4 mL). The resin from each reaction was then allowed to settle and the solvent was removed. The resins were then rinsed three times with methanol (5 mL) and then dried under vacuum.

The dried resins (2 mg) from each reaction were then treated with 0.1 mL of 1:1 dichloromethane and trifluoroacetic acid. After 1 h, the solvent was removed and the dried resins were suspended in 0.2 mL of 1:1 1:1, H₂O/MeCN each containing 0.05% trifluoroacetic acid and filtered. HPLC analysis was performed with the filtrates from each reaction as described in Example 1, section (a). All reactions indicated complete conversion to the products NSP-DMAE-ED, NSP-DMAE-DEG, NSP-DMAE-TEG, NSP-DMAE-TEEG, NSP-DMAE-PEEG and NSP-DMAE-HEG eluting at 10.0 min, 10.2 min, 11.4 min, 12 min, 12.5 min and 12.9 min respectively.

### b) Coupling of Resin-Immobilized NSP-DMAE-spacer to N¹⁰-trifluoroacetyl Pteroic Acid

A solution of N¹⁰-trifluoroacetyl pteroic acid (25 mg, 61.3 μmoles) in anhydrous DMF (1.2 mL) was treated with diisopropylethylamine (13 μL, 1.2 equivalents) and HATU (28 mg, 1.2 equivalents). The reaction was stirred at room temperature for 30 minutes and then 0.2 mL of this solution was added separately to the six resins from section (a). The resins were stirred gently at room temperature. After 16 hours, the reactions were diluted with DMF (2 mL) and methanol (4 mL). After the resins settled, the solvent was removed in each case and the resins were rinsed three times with methanol (5 mL). They were then dried under vacuum. The conjugates were then cleaved from the resins by stirring the resins with 0.5 mL of 1:1 dichloromethane and trifluoroacetic acid at room temperature for one hour. The solvent was then removed from each reaction and the resins were suspended in DMF (2 mL) in each case and filtered. HPLC analysis as described in Example 1, section (a) indicated the conjugates NSP-DMAE-ED-N¹⁰-trifluoroacetyl pteroate, NSP-DMAE-DEG-N¹⁰-trifluoroacetyl pteroate, NSP-DMAE-TEG-N¹⁰-trifluoroacetyl pteroate, NSP-DMAE-TEEG-N¹⁰-trifluoroacetyl pteroate, NSP-DMAE-PEEG-N¹⁰-trifluoroacetyl pteroate and NSP-DMAE-HEG-N¹⁰-trifluoroacetyl pteroate eluting at 15.2 min, 15.2 min, 15.3 min, 15.5 min, 15.7 min and 15.9 min respectively. The conjugates were all purified by preparative HPLC using a C₁₈ 20×300 mm column. The HPLC fraction containing conjugate each case was frozen at −80° C. and lyophilized to dryness.

The lyophilized conjugates were then dissolved in DMF (0.5 mL each) and treated with 0.25 mL of 0.5 M aqueous piperidine at 4° C. The reactions were warmed to room temperature and stirred for one hour. HPLC analysis as described in Example 1, section (a) indicated complete and clean removal of the trifluoroacetyl group in each case to give the conjugates NSP-DMAE-ED-pteroate, NSP-DMAE-DEG-pteroate, NSP-DMAE-TEG-pteroate, NSP-DMAE-TEEG-pteroate, NSP-DMAE-PEEG-pteroate and NSP-DMAE-HEG-pteroate eluting at 13.6 min, 12.9 min, 13.5 min, 13.8 min, 14 min and 14.3 min respectively. The reactions were all frozen at −80° C. and lyophilized to dryness.

The conjugates were also characterized by MALDI-TOF mass spectroscopy as indicated below in Table 4.

TABLE 4

| Conjugate | Observed mass | Calculated mass |
|---|---|---|
| NSP-DMAE-ED-Pteroate | 831.8 | 830.3 |
| NSP-DMAE-DEG-Pteroate | 876.6 | 874.3 |
| NSP-DMAE-TEG-Pteroate | 920.8 | 918.3 |
| NSP-DMAE-TEEG-Pteroate | 965.9 | 962.4 |
| NSP-DMAE-PEEG-Pteroate | 1009.3 | 1006.4 |
| NSP-DMAE-HEG-Pteroate | 1053.3 | 1050.4 |

### Example 3

### Synthesis of Unnatural NSP-DMAE-FOLATE Conjugates on Wang Resin

### a) Synthesis of Resin-Immobilized NSP-DMAE-TEG

Wang resin-immobilized NSP-DMAE-PFP ester (0.9 g resin, 0.38 mg acridinium ester/10 mg resin) was suspended in anhydrous dichloromethane (10 mL) and treated with diamino-TEG (75 uL, 50 mM). The resin was stirred gently at room temperature for 4 hours. The resin was then diluted with ethyl acetate and the resin was allowed to settle. The solvent was removed and the resin was rinsed several times with methanol (5×10 mL) and then dried under vacuum.

A small amount (10 mg) of the resin was stirred with 0.5 mL of 1:1, dichloromethane and trifluoroacetic acid at room temperature for 1 hour. The reaction was then diluted with methanol (1 mL) and then filtered. The filtrate was evaporated to dryness and the acridinium ester residue was dissolved in 1 mL 1:1, H₂O/MeCN each containing 0.05% trifluoroacetic acid. HPLC analysis of this solution as described in Example 1, section (a) showed product NSP-DMAE-TEG eluting at 11.4 minutes (94% purity) and UV-visible spectrophotometric analysis indicated an acridinium ester yield of 85%.

### b) Coupling of Resin-Immobilized NSP-DMAE-TEG to FMOC-Protected Amino Acids

Procedure A: The following procedure illustrated for the coupling of FMOC-leucine-PFP ester to resin-immobilized NSP-DMAE-TEG was used for the coupling of all commercially available FMOC-amino acid-PFP esters (Pro, Phe, Isoleu, Leu, Met, Tyr, Ala, His and Norleu).

Wang resin-NSP-DMAE-TEG from section (a) (25 mg, ˜1 mg acridinium ester) was treated with a DMF (0.5 mL) solution of FMOC-leucine-PFP ester (25 mM, 6.5 mg) and diisopropylethylamine (2 μL). The reaction was stirred gently at room temperature. After 4 hours, the reaction was diluted with DMF (2 mL) and after the resin settled, the solvent was removed. The resin was rinsed with DMF (2 mL) twice followed by methanol (3×3 mL) and then dried under vacuum.

Procedure B: The following procedure illustrated for the coupling of FMOC-valine to resin-immobilized NSP-DMAE-TEG was used for the coupling of all commercially available FMOC-amino acids (Val, Ser, Gln, and Arg).

A solution of FMOC valine (4.3 mg, 12.5 μmoles, 25 mM) in anhydrous DMF (0.4 mL) was treated with diisopropylthylamine (2.2 μL, 12.5 μmoles) and HATU (5.7 mg, 12.5 μmoles). After 5 minutes, this solution was added to resin-immobilized NSP-DMAE-TEG (25 mg, ˜1 mg acridinium ester) in DMF (0.1 mL). The reaction was stirred at room temperature for 16 hours. The reaction was then diluted with DMF (2-3 mL) and after the resin settled, the solvent was removed. The resin was rinsed with DMF (2×3 mL) and then methanol (3×3 mL) followed by drying under vacuum.

### c) Cleavage of the FMOC Group from Resin-Immobilized NSP-DMAE-TEG-Amino Acid

The following general procedure was used.

The resins from (b) were stirred in DMF (0.25 mL) containing 1% piperidine at room temperature. After one hour, the reactions were diluted with ethyl acetate (3 mL) and after the resin settled, the solvent was removed. The resins were rinsed with ethyl acetate and methanol several times and then dried under vacuum.

### d) Coupling of Resin-Immobilized NSP-DMAE-TEG-amino Acid to N¹⁰-trifluoroacetyl Pteroic Acid Followed by Cleavage from the Resin

The following general procedure illustrated for the coupling of resin immobilized NSP-DMAE-TEG-Phe to N¹⁰-trifluoroacetyl pteroic acid was used for all the other unnatural folate conjugates.

A solution of N¹⁰-trifluoroacetyl pteroic acid (4 mg, 9.8 μmoles) in DMF (0.4 mL) was treated with diisopropylethylamine (2 μL, 1.2 equivalents) and HATU (4.5 mg, 1.2 equivalents). The reaction was stirred at room temperature for 30 minutes then was added to resin-immobilized NSP-DMAE-TEG-Phe along with additional diisopropylethylamine (1 μL). The reaction was stirred at room temperature for 16 hours. The reaction was then diluted with DMF (2 mL) and after the resin settled, the solvent was removed. The resin was rinsed with DMF (4×2 mL) and methanol (4×2 mL). It was then dried under vacuum. It was then treated with 0.5 mL of 1:1, dichloromethane and trifluoroacetic acid and stirred for one hour. The reaction was then diluted with DMF (1.5 mL) and filtered to remove the resin. HPLC analysis of the filtrate as described in Example 1, section (a), showed product eluting at 17 minutes. The product was purified by preparative HPLC using a C₁₈, 20×300 mm column. The HPLC fraction containing conjugate each case was frozen at −80° C. and lyophilized to dryness. The HPLC retention times of the other conjugates were Pro (15.3 min), Val (161 min), Arg (13.8 min), Gln (14.1 min), Ser (14.1 min), Leu (17.2 min), Met (16.3 min), Isoleu (17 min), Tyr (15.5 min), Ala (15.1 min), His (13.5 min) and Norleu (17.2 min).

### e) Hydrolysis of the N10-trifluoroacetyl Group in NSP-DMAE-TEG-amino Acid-N¹⁰-trifluoroacetyl Pteroate Conjugates

The following general procedure was employed. The conjugates from (d) were dissolved in DMF (0.5 mL) and treated with 0.3 mL of 0.5 M aqueous piperidine. The reactions were stirred at room temperature for 1 hour and then transferred to a freezer at −15° C. Each reaction was analyzed by HPLC as described in Example 1, section (a). After HPLC analysis, the reaction mixtures were frozen at −80° C. and lyophilized to dryness. The HPLC retention times of the conjugates and the observed molecular ions by MALDI-TOF mass spectroscopy are also included.

TABLE 5

| | HPLC retention | Observed |
|---|---|---|
| Conjugate | time (min) | mass |
| NSP-DMAE-TEG-Arg-Pteroate | 12.4 | 1078.5 9 |
| NSP-DMAE-TEG-His-Pteroate | 12.1 | 1058.8 |
| NSP-DMAE-TEG-Gln-Pteroate | 12.8 | 1048.7 |
| NSP-DMAE-TEG-Ser-Pteroate | 12.5 | 1007.9 |
| NSP-DMAE-TEG-Ala-Pteroate | 13.5 | 992.7 |
| NSP-DMAE-TEG-Pro-Pteroate | 14.0 | 1019.1 |
| NSP-DMAE-TEG-Met-Pteroate | 14.6 | 1052.1 |
| NSP-DMAE-TEG-Val-Pteroate | 14.6 | 1021.0 |
| NSP-DMAE-TEG-Tyr-Pteroate | 14.0 | 1084.7 |
| NSP-DMAE-TEG-Isoleu-Pteroate | 15.3 | 1034.6 |
| NSP-DMAE-TEG-Leu-Pteroate | 15.4 | 1033.9 |
| NSP-DMAE-TEG-Norleu-Pteroate | 15.4 | 1035.1 |
| NSP-DMAE-TEG-Phe-Pteroate | 15.7 | 1069.0 |
| NSP-DMAE-TEG-α-folate | 12.7 | 1050.1 |
| NSP-DMAE-TEG-γ-folate | 12.7 | 1050.2 |

### Example 4

### Solid Phase Synthesis of DMAE Derivatives

### a) Synthesis of 2′,6′-dimethyl-4′-[fluorenylmethyloxycarbonyl]-amidoethylamidocarbonylphenyl-10-N-benzyloxycarbonylmethyl-acridinium-9-carboxylate (NCM-DMAE-ED-FMOC)

A suspension of 2′,6′-dimethyl-4′-N-succinimidyloxycarbonylphenyl-acridine-9-carboxylate (50 mg) in benzyl iodoacetate (3 mL, (synthesized from benzyl bromoacetate and sodium iodide) was heated at 140° C. under a nitrogen atmosphere for 16 hours. It was then cooled to room temperature and poured into hexanes (˜30 mL). A dark brown solid separated out. The hexanes were decanted and the residue was rinsed several times with hexanes. The crude product was then dissolved in MeCN (25 mL) and analyzed by HPLC using an analytical C₁₈ column from Phenomenex, 4.6 mm×30 cm and a 30 minute gradient of 10→100% MeCN/H₂O with 0.05% trifluoroacetic acid at a flow rate of 1 mL/min. and UV detection at 260 nm. Product was observed eluting at 19 minutes. The MeCN solution was concentrated to a small volume and purified by preparative HPLC as described earlier. Evaporation of the HPLC fraction, containing product afforded the product as an oily solid. Yield=55 mg. This material was treated with a DMF solution (2 mL) of FMOC-ED (Aldrich, 52 mg, 0.14 mmol) along with diisopropylethylamine (50 μL, 0.29 mmol). The reaction was stirred at room temperature. After one hour, HPLC analysis indicated product eluting at 20.6 minutes, which gave a molecular ion at 785 by MALDI-TOF mass spectroscopy. The reaction mixture was evaporated to dryness to give a sticky solid. This material was treated with 30% HBr/AcOH and the reaction was stirred at room temperature for 16 hours. Ether (25 mL) was then added and the precipitated solid was collected by filtration and rinsed with ether. The crude material was dissolved in DMF (5 mL) and analyzed by HPLC, which indicated product eluting at 17.4 minutes. The product was isolated by preparative HPLC. The HPLC fraction, containing product was concentrated to a small volume, frozen at −80° C. and lyophilized to dryness. Yield=20 mg.

### b) Immobilization of NCM-DMAE-ED-FMOC onto Polystyrene Wang Resin and Cleavage of the FMOC Group

Polystyrene Wang resin (1.8-2.0 mmol OH/g, 0.1 g) was suspended in 2 mL of 10% DMF, 90% dichloromethane and treated with a solution of NCM-DMAE-ED-FMOC (6.5 mg, 9.4 μmoles) and 1-hydroxybenzotriazole (10 m5, 75 μmoles) in DMF (0.2 mL). Diisopropylcarbodiimide (23 μL, 150 μmoles) was added followed by 4-dimethylaminopyridine (1.2 mg, 0.1 μmol). The reaction was stirred at room temperature for 16 hours. The reaction was then diluted with methanol and after the resin settled, the solvent was removed. The resin was rinsed several times with methanol (5 mL) and dried under vacuum.

The extent of acridinium ester incorporation was determined by treating 8.6 mg of the resin with 0.5 mL of 1:1, dichloromethane and trifluoroacetic acid. The reaction was stirred at room temperature for one hour and then diluted with methanol (3 mL). The reaction was then filtered and the filtrate was evaporated to dryness. The acridinium ester cleaved off the resin was dissolved in 1 mL of 1:1, MeCN and water containing 0.05% trifluoroacetic acid. HPLC analysis of this solution as described in section (a) showed NCM-DMAE-ED-FMOC eluting at 17.5 minutes of 82% purity. UV-Visible spectrophotometric analysis as described in Example 1, section (a) indicated an immobilization efficiency of ˜20% corresponding to 0.1 mg acridinium ester per 8.6 mg resin.

The FMOC group was then cleaved as follows. The resin from above was stirred in DMF (2 mL) containing 1% piperidine at room temperature for 1 hour. The reaction was then diluted with ethyl acetate (5 mL) and after the resin settled, the solvent was removed. The resin was rinsed once more with ethyl acetate and several times with methanol. It was then dried under vacuum. A small amount (5.6 mg) of the resin was stirred with 0.5 mL of 1:1, dichloromethane and trifluoroacetic acid. The reaction was stirred at room temperature for one hour and then diluted with methanol (1 mL). The reaction was then filtered and the filtrate was evaporated to dryness. The acridinium ester cleaved off the resin was dissolved in 1 mL of 1:1, MeCN acid and water containing 0.05% trifluoroacetic acid. HPLC analysis of this solution as described in section (a) showed NCM-DMAE-ED eluting at 9.5 minutes and <5% of the starting material.

### c) Solid Phase Synthesis of Resin-Immobilized NCM-DMAE-ED-Theophylline

The following procedure illustrated for the solid phase synthesis of NCM-DMAE-ED-Theophylline was also used for the synthesis of the estradiol and pteroate conjugates.

A solution of 8-carboxypropyl theophylline (Sigma, 1.6 mg, 6 μmoles) in DMF (0.5 mL) was treated with disopropylethylamine (1.6 uL, 1.5 equivalents) and HATU (2.7 mg, 1.2 equivalents). After 15 minutes at room temperature, this solution was added to 10 mg of the resin from section (b) and the reaction was stirred at room temperature for 16 hours. The resin was then rinsed twice with DMF (3×2 mL) and methanol (4×3 mL). It was then dried under vacuum. The dried resin was stirred with 0.5 mL of 1:1, dichloromethane and trifluoroacetic acid at room temperature for one hour and then diluted with methanol (2 mL). The reaction was then filtered and the filtrate was evaporated to dryness. The conjugate cleaved off the resin was dissolved in 1 mL of 2:1, MeCN acid and water containing 0.05% trifluoroacetic acid. Analysis by UV-Visible spectrophotometry as described in Example 1, section (a) indicated an acridinium ester yield of 97%. HPLC analysis as described in section (a) showed the conjugate eluting at 11.5 minutes as a broad peak, which showed the molecular ion at 721 mass units corresponding to the conjugate when analyzed by MALDI-TOF mass spectroscopy. The solution was evaporated to dryness. The residue was suspended in toluene (˜5 mL) and evaporated to dryness. The product was then heated in acetic acid (0.5 mL) at 85° C. for 3 hours to effect decarboxylation of the NCM group. The reaction was then cooled to room temperature and analyzed by HPLC which indicated the product DMAE-ED-theophylline eluting at 11.9 minutes. MALDI-TOF mass spectroscopy gave the molecular ion at 677 mass units corresponding to the decarboxylated conjugate. The acetic acid solution containing the conjugate was diluted with toluene (5 mL) and evaporated to dryness.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims.

What is claimed is:

1. An acridinium-functionalized solid-phase support comprising a solid phase support having immobilized thereon a chemiluminescent substituted acridinium compound; said substituted acridinium compound comprising a linker group covalently attached to the nitrogen atom of the acridinium nucleus and said solid phase support.

2. The acridinium-functionalized solid-phase support of claim 1 having the structure of formula I: 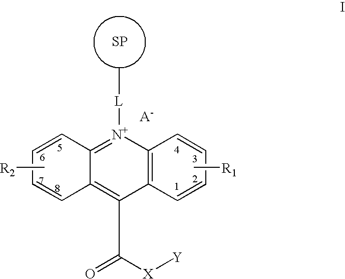
wherein,
L is a sulfonate ester or carboxylate ester linker group between the nitrogen of the acridinium nucleus and said solid phase support;
R₁ represents a substituent at one or more of carbon atoms 1-4 and R₂ represents a substituent at one or more of carbon atoms 5-8; R₁ and R₂ being independently selected at each occurrence from the group consisting of hydrogen, substituted or unsubstituted alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof;
X is O, S, or NR^{a}; where R^{a} is —SO₂—R′, R′ being selected from the group consisting of hydrogen, substituted or unsubstituted, branched or straight chain alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof;
and in the case where X is O or S, Y is a substituent of the formula: 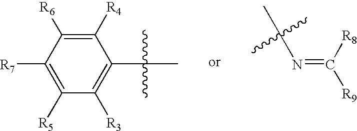
wherein at least one of R₃, R₄, R₅, R₆, R₇, R₈, and R₉ is independently a group -Q-R₁₀, wherein R₁₀ is a group comprising one or more reactive functional groups; where Q represents a bond or a functional group selected from the group consisting of branched or straight-chain alkyl, alkenyl, alkynyl, substituted or unsubstituted aryl, alkyl-aryl, and aryl-alkyl, optionally containing one or more heteroatoms selected from the group consisting of oxygen, nitrogen, phosphorous, sulfur, halogen, and combinations thereof;
and wherein any of R₃, R₄, R₅, R₆, R₇, R₈, and R₉ which are not a group -Q-R₁₀ are substituents independently selected from the group consisting of substituents defined above for R₁ and R₂, hydrogen, hydroxyl, halogen, alkyl, alkenyl, alkynyl, aryl, alkyl-aryl, or aryl-alkyl, —OR^{b}, —SR^{b}, cyano, carboxyl, —(C═O)—OR^{b}, —NR^{b}R^{c}, or —(C═O)—NR^{b}R^{c}, where R^{b} and R^{c} are independently selected from the substituents defined above for R₁ and R₂;
and in the case where X is NR^{a}, Y is a group -Q-R₁₀ as defined above;
A⁻ is a counter ion selected from the group consisting of CH₃SO₄⁻, FSO₃⁻, CF₃SO₄⁻, C₄F₉SO₄⁻, CH₃C₆H₄SO₃⁻, halide, CF₃COO⁻, CH₃COO⁻, and NO₃⁻; and
SP represents a solid phase support selected from the group consisting of polystyrene Wang resin, a paramagnetic particle, a latex particle, and a microtiter plate.

3. The acridinium-functionalized solid-phase support of claim 2, wherein R₁₀ comprises one or more nucleophilic groups, electrophilic groups, and combinations thereof.

4. The acridinium-functionalized solid-phase support of claim 3, wherein R₁₀ comprises one or more nucleophilic groups selected from the group consisting of amino, hydroxyl, sulfhydryl, sodium or lithium organic metallic moieties, or an active methylene group adjacent to a strong electron-withdrawing group; such electron-withdrawing groups consisting of —NO₂, —CN, —SO₃H, —N(R*)₃⁺, and —S(R*)₃⁺, wherein R* is selected from the group consisting of hydrogen, substituted or unsubstituted, branched or straight chain alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof.

5. The acridinium-functionalized solid-phase support of claim 3, wherein R₁₀ represents a group selected from the group consisting of: 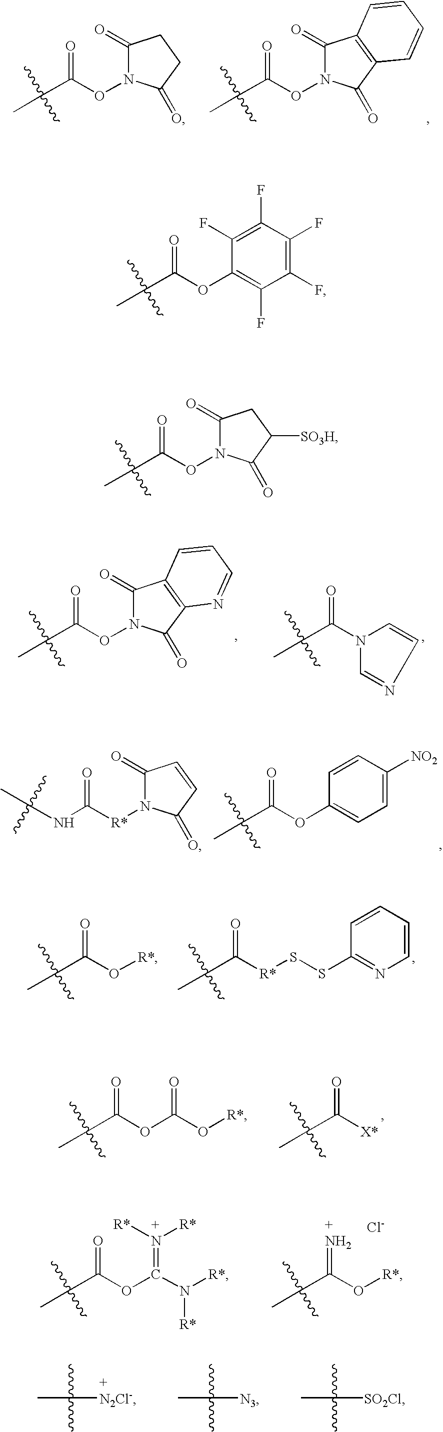 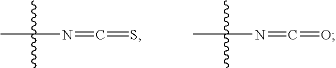
wherein X* is a halogen; and R* is a functional group selected from the group consisting of hydrogen, substituted or unsubstituted, branched or straight chain alkyl, alkenyl, alkynyl, aryl, alkyl-aryl, or aryl-alkyl, and combinations thereof, optionally containing one or more heteroatoms selected from the group consisting of oxygen, nitrogen, phosphorous, sulfur, halogen, and combinations thereof.

6. The acridinium-functionalized solid-phase support of claim 2, wherein L is a sulfonate ester of the form —(CH₂)ₙ—S(═O)₂—O—, where n is 3 or 4.

7. The acridinium-functionalized solid-phase support of claim 6 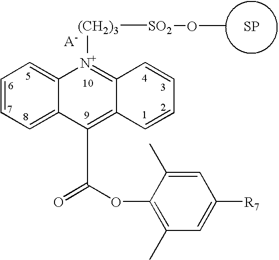
having the following structure:
wherein A⁻, R₇ and SP are defined as:
A⁻ is a counter ion selected from the group consisting of CH₃SO₄⁻FSO₃⁻, CF₃SO₄⁻, CH₃C₆H₄SO₃⁻, halide CF₃COO⁻, CH₃COO⁻, and NO₃⁻; and
R₇ is independently a group -Q-R₁₀ wherein R₁₀ is a group comprising one or more reactive functional groups; where Q represents a bond or a functional group selected from the group consisting of branched or straight-chain alkyl, alkenyl, alkynyl, substituted or unsubstituted aryl, alkyl-aryl, and aryl-alkyl, optionally containing one or more heteroatoms selected from the group consisting of oxygen, nitrogen, phosphorous, sulfur, halogen, and combinations thereof; and
SP represents a solid phase support selected from the group consisting of polystyrene Wang resin, a paramagnetic particle, a latex particle, and a microtiter plate.

8. The acridinium-functionalized solid-phase support of claim 7 having the following structure: 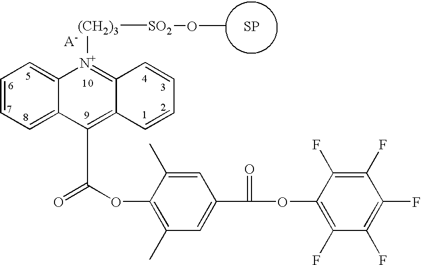
wherein A⁻ and SP are defined as:
A⁻ is a counter ion selected from the group consisting of CH₃SO₄⁻, FSO₃⁻, CF₃SO₄⁻, C₄F₉SO₄⁻, CH₃C₆H₄SO₃⁻, halide, CF₃COO⁻, CH₃COO⁻, and NO₃⁻; and
SP represents a solid phase support selected from the group consisting of polystyrene Wang resin, a paramagnetic particle, a latex particle, and a microtiter plate.

9. The acridinium-functionalized solid-phase support of claim 2, wherein L is a carboxylate ester of the form —CH₂—(C═O)—O—.

10. The acridinium-functionalized solid-phase support of claim 9 having the following structure: 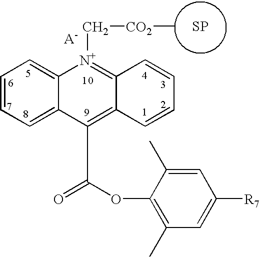
wherein A⁻, R₇ and SP are defined as:
A⁻ is a counter ion selected from the group consisting of CH₃SO₄⁻, FSO₃⁻, CF₃ SO₄⁻, C₄F₉SO₄⁻, CH₃C₆H₄SO₃⁻, halide, CF₃COO⁻, CH₃COO⁻, and NO₃⁻; and
R₇ is independently a group -Q-R₁₀, wherein R₁₀ is a group comprising one or more reactive functional groups; where Q represents a bond or a functional group selected from the group consisting of branched or straight-chain alkyl alkenyl, alkynyl, substituted or unsubstituted aryl, alkyl-aryl, and aryl-alkyl, optionally containing one or more heteroatoms selected from the group consisting of oxygen, nitrogen, phosphorous, sulfur, halogen, and combinations thereof; and
SP represents a solid phase support selected from the group consisting of polystyrene Wang resin, a paramagnetic particle, a latex particle, and a microtiter plate.

11. The acridinium-functionalized solid-phase support of claim 10 having the following structure: 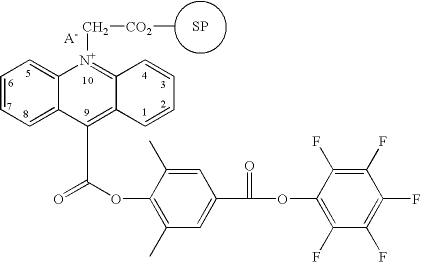
wherein SP and A⁻ are defined as:
SP represents a solid phase support selected from the group consisting of polystyrene Wang resin, a paramagnetic particle, a latex particle, and a microtiter plate; and
A⁻ is a counter ion selected from the group consisting of CH₃SO₄, FSO₃⁻, CF₃SO₄⁻, C₄F₉SO₄⁻, CH₃C₆H₄SO₃⁻, halide, CF₃COO⁻, CH₃COO⁻, and NO₃⁻.

12. The acridinium-functionalized solid-phase support of claim 2 wherein Q is the group —(C═O)—NH—R₁₁—NH—R₁₂—, where R₁₁ and R₁₂ are independently selected from the group consisting of alkyl, alkenyl, alkynyl, aryl, aryl-alkyl, and alkyl-aryl, optionally containing one or more heteroatoms selected from the group consisting of oxygen, nitrogen, phosphorous, sulfur, halogen, and combinations thereof.

13. The acridinium-functionalized solid-phase support of claim 12 wherein Q is a group —NH—(CH₂CH₂O)ₙ—CH₂CH₂NH—C(O)(CH₂)ₘ— where n=0-20 and m=1-4.

14. A method of solid phase synthesis of acridinium compound derivatives or conjugates comprising the steps of:
(a) providing an acridinium-functionalized solid phase support comprising a solid phase support having immobilized thereon a chemiluminescent substituted acridinium compound; said substituted acridinium compound comprising a linker group covalently attached to the nitrogen atom of the acridinium nucleus and said solid phase support;
(b) performing one or more synthetic transformations on said acridinium compound to provide a derivative or conjugate of said acridinium compound;
(c) cleaving said derivative or conjugate of said acridinium compound from said solid phase support.

15. A method of solid phase synthesis of acridinium compound derivatives or conjugates comprising the steps of:
(a) providing an acridinium-functionalized solid phase support of any of claims 2-13;
(b) performing one or more synthetic transformations on said acridinium compound to provide a derivative or conjugate of said acridinium compound;
(c) cleaving said derivative or conjugate of said acridinium compound from said solid phase support.

16. The method of claim 14, wherein said linker is a N-sulfoalkyl group.

17. The method of claim 14, wherein said linker is a N-carboxymethyl group.

18. The method of claim 17 further comprising the step of decarboxylating said derivative or conjugate of said acridinium compound by heating in acetic acid.

19. The method of claim 14 wherein said derivative or conjugate of said acridinium compound is a conjugate of said acridinium compound with a biologically active molecule.

20. The method of claim 19 wherein said biologically active molecule is selected from the group consisting of steroids, vitamins, hormones, therapeutic drugs, peptides and nucleic acids.
